(19) United States
(12) Patent Application Publication
(10) Pub. No.: US 2009/0189120 A1
(43) Pub. Date: Jul. 30, 2009

(54) COMPOUND, LIQUID CRYSTAL COMPOSITION, AND ANISOTROPIC MATERIAL

(75) Inventor: Hiroshi TAKEUCHI, Ashigarakami-gun (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Minato-ku (JP)

(21) Appl. No.: 12/361,097

(22) Filed: Jan. 28, 2009

(30) Foreign Application Priority Data
Jan. 29, 2008 (JP) 2008-017125

(51) Int. Cl. C09K 19/34; C07D 409/04; C07D 333/52

(52) U.S. Cl. 252/299.61; 548/364.4; 549/32; 544/302

(57) ABSTRACT

A compound represented by formula (I) below:

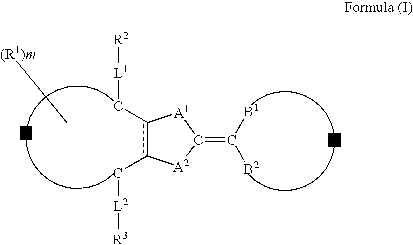

where, each of A¹ and A² independently represents a group selected from the group consisting of —O—, —NR— (R represents a hydrogen atom or substituent), —S— and —CO—; Z forms a five- or six-membered ring together with C—C═C—C or C═C—C═C illustrated in the formula; Y forms a five- to seven-membered ring together with B¹—C—B² illustrated in the formula, provided that those having the same substituent for A¹ and B¹ and for A² and B² and those having the same substituent for A¹ and B² and for A² and B¹ are excluded, is disclosed.

### CROSS-REFERENCE TO RELATED APPLICATIONS

This application claims benefit of priority under 35 U.S.C. 119 to Japanese Patent Application No. 2008-017125 filed on Jan. 29, 2008, which is expressly incorporated herein by reference in its entirety.

### BACKGROUND OF THE INVENTION

1. Field of the Invention

The present invention relates to a novel compound, a liquid crystal composition containing the same, and an anisotropic material obtained by fixing alignment of the liquid crystal composition.

2. Related Art

So-called retardation plates publicly known include thin plates composed of an inorganic material such as calcite, mica, quartz or the like, and stretched polymer films having large intrinsic birefringence. The retardation plates may be categorized into ¼ wave plate (abbreviated as “λ/4 plate”, hereinafter) converting linear polarization into circular polarization, and ½ wave plate (abbreviated as “λ/2 plate”, hereinafter) causing 90° conversion of the plane of polarization oscillation of linear polarization. The conventional retardation plates are capable of adjusting retardation of monochromatic light to ¼ or ½ of the wavelength thereof, but a problem arises in that white light, which is a synthetic light having rays in the visible region mixed therein, may be converted only into a colored polarized light, due to distribution of the state of polarization at the individual wavelength. This is ascribable to that the material composing the retardation plate has wavelength dispersion characteristics of retardation.

In order to solve this problem, various types of wide-band retardation plate, capable of providing uniform retardation over a wide wavelength region, have been investigated (for example, Japanese Unexamined Patent Publication (KOKAI, occasionally referred to as “JPA”) Nos. Heisei 10-68816, Heisei 10-90521, Heisei 11-52131, 2000-284126, 2001-4837 and WO00/2675 pamphlet).

In recent pursuit of thinner retardation plates used in reflective liquid crystal display devices, and aiming at realizing a wide-band λ/4 plate, there has been proposed also a liquid crystal composition having reversed wavelength dispersion characteristics of retardation (for example, Japanese Unexamined Patent Publication Nos. 2002-267838, 2003-160540, 2005-208414, 2005-208415, 2005-208416, 2005-289980, and 2006-330710). In particular, a method of producing a retardation plate using a composition containing a low-molecular-weight liquid crystalline compound may be effective for thinning the retardation plate. The low-molecular-weight liquid crystalline compound applicable to such purpose often has a molecular structure capable of absorbing light in longer wavelength region, in the direction lateral to the longitudinal axis of the molecule. Although the colored retardation plate has conventionally been limited in the application to a considerable degree, recent techniques of patterning the retardation plate have made it possible to use, for example, a retardation plate colored in yellow also in green and red display regions as a result of patterning. The conventional wide-band λ/4 wave plate using a liquid crystal composition having reversed wavelength dispersion characteristics of retardation is, however, not so satisfactory in the characteristics particularly in the longer wavelength region, and has been remained to be further improved.

### SUMMARY OF THE INVENTION

It is therefore an object of the present invention to provide a novel compound exhibiting reversed wavelength dispersion characteristics of Δn, and improved in reversed wavelength dispersion characteristics of Δn particularly in longer wavelength region; a liquid crystal composition containing the compound; and an optically anisotropic material using the liquid crystal composition.

The means for achieving the above mentioned objects are as follows.
[1] A compound represented by formula (I) below:

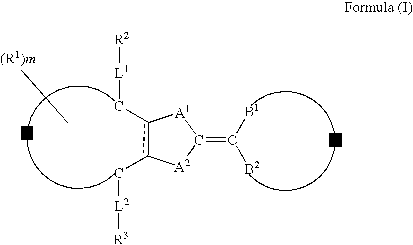

where, each of A¹ and A² independently represents a group selected from the group consisting of —O—, —NR— (R represents a hydrogen atom or substituent), —S— and —CO—; Z represents one or two atoms selected from the group consisting of carbon atom and the Group XIV to Group XVI non-metallic atoms, and forms a five- or six-membered ring together with C—C═C—C or C═C—C═C illustrated in the formula; each of R¹, R² and R³ independently represents a substituent; m represents an integer from 0 to 4; each of L¹ and L² independently represents a single bond or divalent linking group; each of B¹ and B² independently represents a group selected from the group consisting of —O—, —S—, —SO—, —SO₂—, —NR— (R represents a hydrogen atom or substituent), ═N—, ═N⁽⁺⁾R— (R represents a hydrogen atom or substituent), —CO—, —CS—, and ═CR— (R represents a hydrogen atom or substituent); Y represents two to four atoms selected from the group consisting of carbon atom and the Group XIV to Group XVI non-metallic atoms, and forms a five- to seven-membered ring together with B¹—C—B² illustrated in the formula, allowing thereon bonding of substituent R⁴; provided that those having the same substituent for A¹ and B¹ and for A² and B² and those having the same substituent for A¹ and B² and for A² and B¹ are excluded.
[2] The compound as set forth in [1], represented by formula (I)′ below:

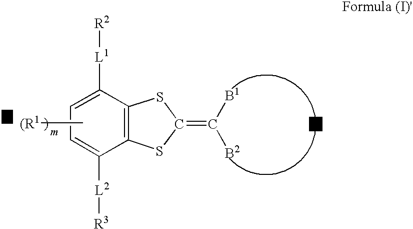

where, S represents a sulfur atom, and definitions of all other symbols are same as those defined in formula (I).
[3] The compound as set forth in [1] or [2], wherein the ring Y is represented by any of formulae Y-1 to Y-24 below:

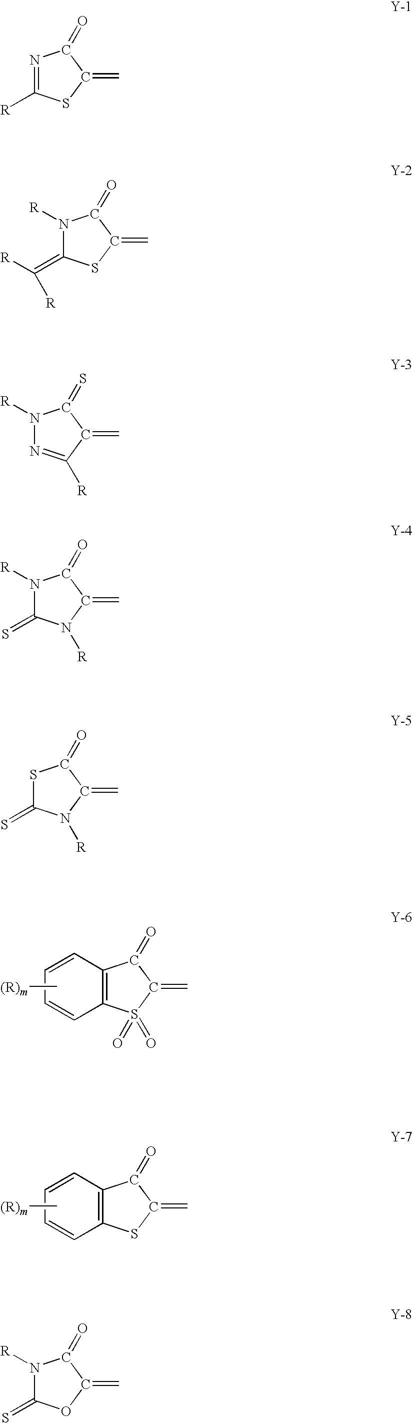

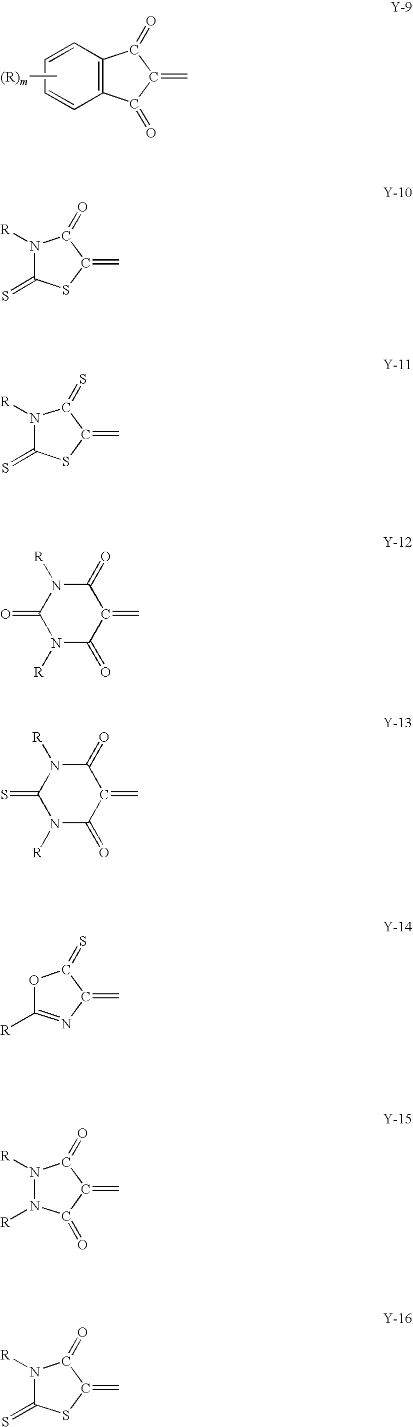

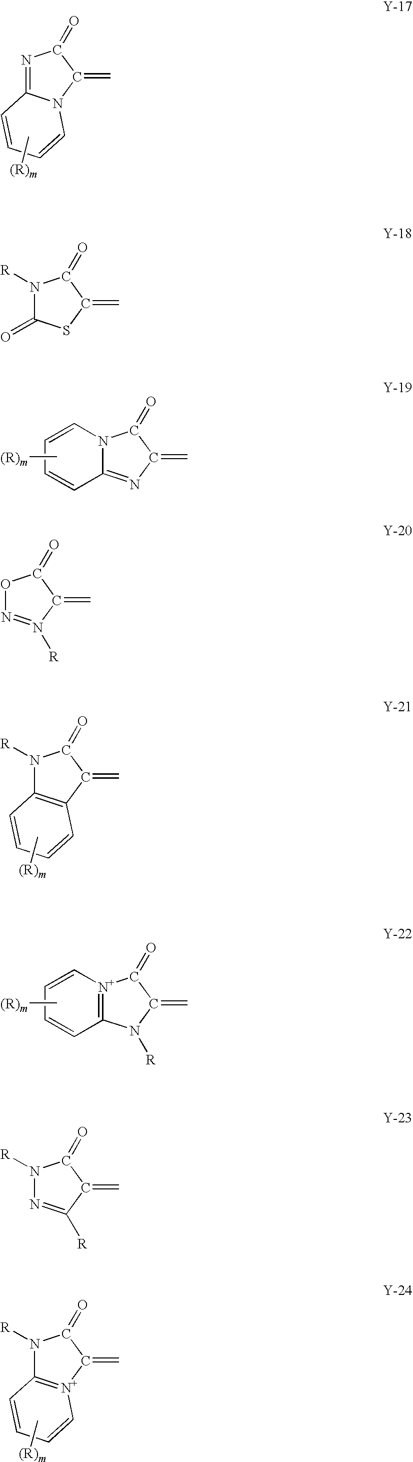

where, R represents a hydrogen atom or substituent; a plurality of (R)s, if coexist therein, may be same with, or different from each other; and m represents an integer from 0 to 4.
[4] The compound as set forth in [1], represented by any one of the formulae (I-a) to (I-e) below:

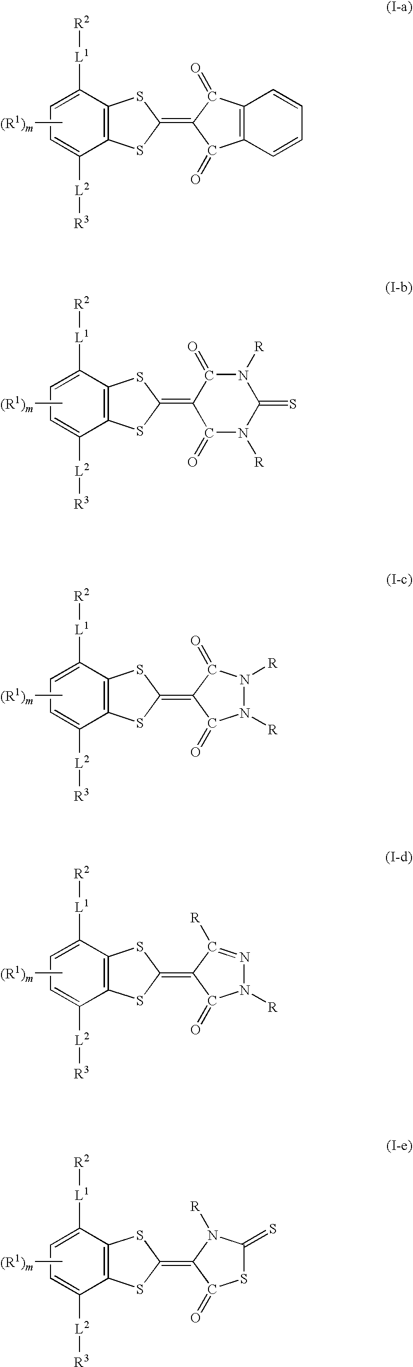

where, S represents a sulfur atom, definitions of all other symbols are same as those defined in the formula (I), R represents a hydrogen atom or substituent; and a plurality of (R)s, if coexist therein, may be same with, or different from each other.
[5] The compound as set forth in any one of [1] to [4], wherein at least either one of L¹ and L² in formula (i) represents —OCO—.
[6] The compound as set forth in any one of [1] to [5], wherein at least one of R, R¹, R², R³ and R⁴ in formula (I) represents a substituent containing polymerizable group(s).
[7] The compound as set forth in any one of [1] to [6], wherein each of R² and R³ is represented by any one of the formulae below:

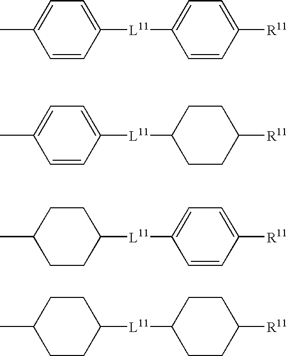

where, L¹¹ represents a single bond or linking group; and R¹¹ represents a substituent.
[8] The compound as set forth in any one of [1 ] to [7], wherein each of R² and R³ is represented by any one of the formulae below:

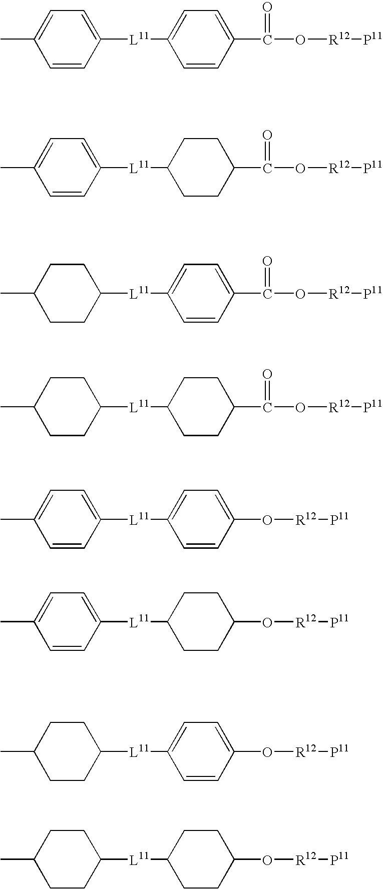

where, L¹¹ has a same definition defined as in the above; R¹² represents an alkyl group (where, either one of non-adjacent carbon atoms may be substituted by an oxygen atom or sulfur atom); and P¹¹ represents a polymerizable group.
[9] A liquid crystal composition comprising at least one species of a compound as set forth in any one of [1] to [8].
[10] An optically anisotropic material formed of a composition as set forth in [9].

### DETAILED DESCRIPTION OF THE INVENTION

The present invention will be detailed below. It is to be understood that any numerical ranges written in a style of “. . . to . . . ” mean the ranges covering the numerical values given before and after “to”, as the lower limit and the upper limit, respectively.

### [Definitions of Re(λ) and Rth(λ)]

In the description, Re(λ) and Rth(λ) each indicate a retardation in plane (unit:nm) and a retardation along thickness direction (unit:nm) at a wavelength λ. Re(λ) is measured by applying a light having a wavelength of λ nm in the normal line direction of a sample such as a film, using KOBRA-21ADH or WR (by Oji Scientific Instruments).

When the sample to be tested is represented by an uniaxial or biaxial refractive index ellipsoid, then its Rth(λ) is calculated according to the method mentioned below.

With the in-plane slow axis (determined by KOBRA 21ADH or WR) taken as the inclination axis (rotation axis) of the sample (in case where the sample has no slow axis, the rotation axis of the sample may be in any in-plane direction of the sample), Re(λ) of the sample is measured at 6 points in all thereof, up to +50° relative to the normal line direction of the sample at intervals of 10°, by applying a light having a wavelength of λ nm from the inclined direction of the sample.

With the in-plane slow axis from the normal line direction taken as the rotation axis thereof, when the sample has a zero retardation value at a certain inclination angle, then the symbol of the retardation value of the sample at an inclination angle larger than that inclination angle is changed to a negative one, and then applied to KOBRA 21ADH or WR for computation.

With the slow axis taken as the inclination axis (rotation axis) (in case where the sample has no slow axis, the rotation axis of the sample may be in any in-plane direction of the film), the retardation values of the sample are measured in any inclined two directions; and based on the data and the mean refractive index and the inputted thickness of the sample, Rth may be calculated according to the following formulae (1) and (2):

$$\begin{matrix}Re ( θ ) = [ nx - \frac{ny \times nz}{\sqrt{\begin{matrix}{\{ ny sin ( {sin}^{- 1} ( \frac{sin ( - θ )}{nx} ) ) \}}^{2} + \\ {\{ nz cos ( {sin}^{- 1} ( \frac{sin ( - θ )}{nx} ) ) \}}^{2}\end{matrix}}} ] \times \frac{d}{cos \{ {sin}^{- 1} ( \frac{sin ( - θ )}{nx} ) \}} & ( 1 ) \\ Rth = \{ ( nx + ny ) / 2 - nz \} \times d & ( 2 )\end{matrix}$$

wherein Re(θ) means the retardation value of the sample in the direction inclined by an angle θ from the normal line direction; nx means the in-plane refractive index of the sample in the slow axis direction; ny means the in-plane refractive index of the sample in the direction vertical to nx; nz means the refractive index of the sample vertical to nx and ny; and d is a thickness of the sample.

When the sample to be tested can not be represented by a uniaxial or biaxial index ellipsoid, or that is, when the sample does not have an optical axis, then its Rth(λ) may be calculated according to the method mentioned below.

With the in-plane slow axis (determined by KOBRA 21ADH or WR) taken as the inclination axis (rotation axis) of the sample, Re(λ) of the sample is measured at 11 points in all thereof, from −50° to +50° relative to the normal line direction of the sample at intervals of 100, by applying a light having a wavelength of λ nm from the inclined direction of the sample. Based on the thus-determined retardation data of Re(λ), the mean refractive index and the inputted thickness of the sample, Rth(λ) of the sample is calculated with KOBRA 21ADH or WR.

The mean refractive index may be used values described in catalogs for various types of optical films. When the mean refractive index has not known, it may be measured with Abbe refractometer. The mean refractive index for major optical film is described below: cellulose acetate (1.48), cycloolefin polymer (1.52), polycarbonate (1.59), polymethylmethacrylate (1.49), polystyrene (1.59).

The mean refractive index and the film thickness are inputted in KOBRA 21ADH or WR, nx, ny and nz are calculated therewith. From the thus-calculated data of nx, ny and nz, Nz=(nx−nz)/(nx−ny) is further calculated.

### [Definition of Δn]

Δn is a value expressing anisotropy of a sample with respect to polarized light, as detailed in “Ekisho Binran (Handbook of Liquid Crystal)”, published by Maruzen Co., Ltd., 2000, Chapter 2, Section 2.3 “Bussei (Physical Characteristics)”, and is defined by difference between a refractive index (nₒ) observed in the direction of polarization normal to the axis of anisotropy, and a refractive index (nₑ) observed in the direction of polarization parallel to the axis of anisotropy, when linear polarized light passes through a substance showing uniaxial anisotropy.

### [Definition of Wavelength Dispersion Characteristics of Δn]

Δn generally varies depending on measurement wavelength. This is ascribable to the fact that the above-described nₒ and nₑ vary depending on wavelength, so that wavelength dispersion characteristics of Δn represents wavelength dependence of difference between refractive indices nₒ and nₑ. Details are described in “Ekisho Binran (Handbook of Liquid Crystal)”, published by Maruzen Co., Ltd., 2000), Chapter 2, Section 2.4.13. In the present invention, expressions of Δn(450), Δn(550) and Δn(650) mean Δn observed at 450 nm, 550 nm and 650 nm, respectively. Note that each measurement wavelength allows an error of ±10 nm.

Methods of measuring Δn of liquid crystal may be exemplified by a method using a wedge-form liquid crystal cell, as described in “Ekisho Binran (Handbook of Liquid Crystal)”, published by Maruzen Co., Ltd., 2000, Chapter 2, Section 2.4.13, for example. According to this method, Δn at the individual wavelengths may be determined by using three types of band pass filters of 450 nm, 550 nm and 650 nm.

Liquid crystal compounds having polymerizable groups may sometimes cause polymerization reaction in the wedge-form liquid crystal cell, and such polymerization often makes the measurement difficult. In this case, the measurement may preferably be carried out by adding a polymerization inhibitor. Alternatively, Δn may be determined (based on an equation Δn=Re/d (thickness)), by measuring retardation (Re) at the individual wavelengths using an instrument capable of measuring retardation, such as KOBRA (trade name, from Oji Scientific Instruments), while keeping the liquid crystal uniformly aligned, and by separately measuring the thickness.

### [Definition of “Regular” and “Reversed” in Wavelength Dispersion Characteristics of Δn]

Substance generally increases its refractive index as wavelength becomes shorter. Also liquid crystal compounds follow this rule, and increases their refractive indices as the measurement wavelength becomes shorter. However, Δn is defined as difference between nₒ and nₑ, so that the wavelength dispersion characteristics of Δn may sometimes be larger in shorter wavelength region, and may sometimes be smaller in the shorter wavelength region, if nₒ and nₑ follow different patterns of wavelength dispersion characteristics. In general, the majority of cases shows a larger value in the shorter wavelength region, and such cases are characterized as “regular wavelength dispersion”. Conversely, the cases showing smaller wavelength dispersion characteristics of Δn in the shorter wavelength region are characterized as “reversed wavelength dispersion”.

### [Definition of Wavelength Dispersion Characteristics of Retardation of Optically Anisotropic Material]

As for optical compensation materials, optical anisotropy is expressed by Re or Rth, wherein definition of which are similar to as described in the above. Conforming to the definition of wavelength dispersion characteristics of Δn, and to the definitions of regular dispersion and reversed dispersion with respect to the wavelength dispersion characteristics of Δn, wavelength dispersion characteristics of Re(λ) or Rth(λ) will be defined as wavelength dispersion characteristics of retardation, wherein the cases showing larger wavelength dispersion characteristics in the shorter wavelength region are characterized as “regular wavelength dispersion”, and the cases showing smaller values in the shorter wavelength region are characterized as “reversed wavelength dispersion”.

First, a compound of the present invention represented by formula (I) will be explained.

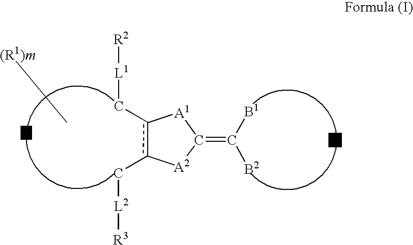

In the formula, where, each of A¹ and A² independently represents a group selected from the group consisting of —O—, —NR— (R represents a hydrogen atom or substituent), —S— and —CO—. Z represents one or two atoms selected from the group consisting of carbon atom and the Group XIV to Group XVI non-metallic atoms, and forms a five- or six-membered ring together with C—C═C—C or C═C—C═C illustrated in the formula. Each of R¹, R² and R³ independently represents a substituent, and m represents an integer from 0 to 4. Each of L¹ and L² independently represents a single bond or divalent linking group. Each of B¹ and B² independently represents a group selected from the group consisting of —O—, —S—, —SO—, —SO₂—, —NR— (R represents a hydrogen atom or substituent), ═N—, ═N⁽⁺⁾R— (R represents hydrogen atom or substituent), —CO—, —CS—, and ═CR— (R represents a hydrogen atom or substituent). Y represents two to four atoms selected from the group consisting of carbon atom and the Group XIV to Group XVI non-metallic atoms, and forms a five- to seven-membered ring together with B¹—C—B² illustrated in the formula. (Substituent R⁴ may be bound thereto.) However, any compounds having the same substituent for A¹ and B¹ and for A² and B² are excluded and any compounds having the same substituent for A¹ and B² and for A² and B¹ are also excluded.

In formula (I), the divalent linking groups represented by L¹ and L² are not specifically limited, but preferably exemplified by the examples below. Note that the left side of each linking group exemplified below corresponds to the position of bonding with the five- or six-membered ring formed by Z together with C—C═C—C or C═C—C═C.

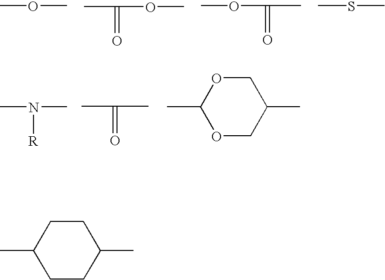

Among these, —O—, —COO— and —OCO— are still more preferable.

In formula (I), Z represents one or two atoms selected from the group consisting of carbon atom and the Group XIV to Group XVI non-metallic atoms, and forms a five- or six-membered ring together with C—C═C—C or C═C—C═C illustrated in the formula. The five- or six-membered ring formed by Z together with C—C═C—C or C═C—C═C is not specifically limited, but preferably exemplified by the examples below. Note that, in the examples below, each broken line expresses the positions of bonding with L¹ or L².

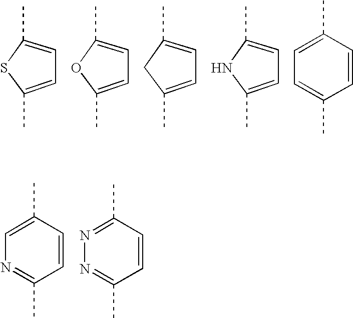

The ring formed by Z together with C—C═C—C or C═C—C═C is preferably a six-membered ring. By selecting the six-membered ring, the compound may be aligned with a higher order-degree. In terms of the same, the ring is also preferably an aromatic ring, and more preferably is a six-membered aromatic ring.

In terms of the above mentioned reason and the synthesis, the ring formed by Z together with C—C═C—C or C═C—C═C is preferably a thiophene ring, benzene ring or pyridine ring, and most preferably a benzene ring.

In formula (I), R¹ is a substituent. When there are a plurality of (R¹)s, such (R¹)s may be same with or different from each other, and may form a ring. Examples of the substituent include halogen atoms such as fluorine atom, chlorine atom, bromine atom and iodine atom; substitute or non-substituted alkyls (preferably, substituted or non-substituted, linear or branched, C₁₋₃₀ alkyls) such as methyl, ethyl, n-propyl, isopropyl, tert-butyl, n-octyl and 2-ethylhexyl; substituted or non-substituted cycloalkyls (preferably substituted or non-substituted C₃₋₃₀ cycloalkyls) such as cyclohexyl, cyclopentyl, and 4-n-dodecylcyclohexyl; substituted or non-substituted bicycloalkyls (preferably substituted or non-substituted C₅₋₃₀ bicycloalkyls, in other words, monovalent residues of C₅₋₃₀ bicycloalkenes removed a hydrogen atom therefrom) such as bicyclo[1,2,2]heptane-2-yl and bicyclo[2,2,2]octane-3-yl; alkenyls (preferably substituted or non-substituted C₂₋₃₀ alkenyls) such as vinyl and allyl; cycloalkenyls (preferably substituted or non-substituted C₃₋₃₀ cycloalkenyls, or, in other words, monovalent residues of C₃₋₃₀ cycloalkens removed a hydrogen atom therefrom) such as 2-cyclopenetne-1-yl and 2-cyclohexene-1-yl; bicycloalkenyls (preferably substituted or non-substituted C₅₋₃₀ bicycloalkenyls, or, in other words, monovalent residues of C₅₋₃₀ bicycloalkenes removed a hydrogen atom therefrom) such as bicyclo[2,2,1]hepto-2-en-1-yl and bicyclo[2,2,2]octo-2-en-4-yl; alkynyls (preferably substituted or non-substituted C₂₋₃₀ alkynyls) such as ethynyl and propargyl; aryls (preferably substituted or non-substituted C₆₋₃₀ aryls) such as phenyl, p-tolyl and naphthyl; heterocyclic groups (preferably residues of 5- or 6-membered, substituted or non-substituted, aromatic or non-aromatic heterocyclic compounds removed a hydrogen atom therefrom, and more preferably 5- or 6-membered C₃₋₃₀ aromatic heterocyclic groups) such as 2-fruryl, 2-thienyl, 2-pyrimidinyl and 2-benzothiazolyl; cyano; hydroxyl; nitro; carboxyl; alkoxys (preferably substituted or non-substituted C₁₋₃₀ alkoxys) such as methoxy, ethoxy, isopropoxy, tert-butoxy, n-octyloxy and 2-methoxy ethoxy; aryloxys (preferably substituted or non-substituted C₆₋₃₀ aryloxys) such as phenoxy, 2-methylphenoxy, 4-tert-butylphenoxy, 3-nitrophenoxy and 2-tetradecanoyl aminophenoxy; silyloxy groups (preferably C₃₋₂₀ silyloxy groups) such as trimethyl silyloxy and tert-butyidimethyl silyloxy; heterocyclic-oxy groups (preferably substituted or non-substituted C₂₋₃₀ heterocyclic-oxy groups) such as 1-phenyl tetrazole-5-oxy and 2-tetrahydro pyranyloxy; acyloxys (preferably formyloxy, substituted or non-substituted C₂₋₃₀ alkylcarbonyloxys and substituted or non-substituted C₆₋₃₀ arylcarbonyloxys) such as formyloxy, acetyloxy, pivaloyloxy, stearoyl oxy, benzoyloxy, and p-methoxyphenyl carbonyloxy; carbamoyloxys (preferably substituted or non-substituted C₁₋₃₀ carbamoyloxys) such as N,N-dimethyl carbamoyloxy, N,N-diethyl carbamoyloxy, morpholino carbonyloxy, N,N-di-n-octylamino carbonyloxy and N-n-octylcarbamoyloxy; alkoxycarbonyloxys (preferably substituted or non-substituted C₂₋₃₀ alkoxycarbonyloxys) such as methoxycarbonyloxy, ethoxycarbonyloxy, tert-butoxycarbonyloxy and n-octylcarbonyloxy; aryloxycarbonyloxys (preferably substituted or non-substituted C₇₋₃₀ aryloxycarbonyloxys) such as phenoxycarbonyloxy, p-methoxyphenoxycarbonyloxy and p-n-hexadecyloxy phenoxycarbonyloxy; aminos (preferably non-substituted amino, substituted or non-substituted C₁₋₃₀ alkylaminos and substituted or non-substituted C₆₋₃₀ anylinos) such as non-substituted amino, methylamino, dimethylamino, anilino, N-methyl-anilino and diphenylamino; acylaminos (preferably formylamino, substituted or non-substituted C₁₋₃₀ alkylcarbonylaminos and substituted or non-substituted C₆₋₃₀ arylcarbonylaminos) such as formylamino, acetylamino, pivaloylamino, lauroylamino and benzoylamino; aminocarbonylaminos (preferably substituted or non-substituted C₁₋₃₀ aminocarbonylaminos) such as carbamoylamino, N,N-dimethylamino carbamoylamino, N,N-diethylamino carbamoylamino and morpholino carbamoylamino; alkoxycarbonylaminos (preferably substituted or non-substituted C₂₋₃₀ alkoxycarbonylaminos) such as methoxycarbonylamino, ethoxycarbonylamino, tert-butoxycarbonylamino, n-octadecyloxy carbonylamino and N-methyl-methoxycarbonylamino; aryloxycarbonylaminos (preferably substituted or non-substituted C₇₋₃₀ aryloxycarbonylaminos) such as phenoxycarbonylamino, p-chlorophenoxycarbonylamino and m-n-octyloxy phenoxycarbonyl amino; sulfamoylaminos (preferably substituted or non-substituted C₀₋₃₀ sulfamoylaminos) such as sulfamoylamino, N,N-dimethylamino sulfonylamino and N-n-octylamino sulfonylamino; alkyl- and aryl-sulfonylaminos (preferably substituted or non-substituted C₁₋₃₀ alkylsulfonylaminos and substituted or non-substituted C₆₋₃₀ arylsulfonylaminos) such as methylsulfonylamino, butylsulfonylamino, phenylsulfonylamino, 2,3,5-trichlorophenyl sulfonylamino and p-methylphenyl sulfonylamino; mercapto; alkylthios (preferably substituted or non-substituted C₁₋₃₀ alkylthios) such as methylthio, ethylthio and n-hexadecylthio; arylthios (preferably substituted or non-substituted C₆₋₃₀ arylthios) such as phenylthio, p-chlorophenylthio and m-methoxyphenylthio; heterocyclicthios (preferably substituted or non-substituted C₂₋₃₀ heterocyclicthios) such as 2-benzothiazolylthio and 1-phenyltetrazol-5-ylthio; sulfamoyls (preferably substituted or non-substituted C₀₋₃₀ sulfamoyls) such as N-ethylsulfamoyl, N-(3-dodecyloxypropyl)sulfamoyl, N,N-dimethylsulfamoyl, N-acetylsulfamoyl, N-benzoylsulfamoyl and N-(N′-phenylcarbamoyl)sulfamoyl; sulfo; alkyl- and aryl-sulfinyls (preferably substituted or non-substituted C₁₋₃₀ alkylsulfinyls and substituted or non-substituted C₆₋₃₀ arylsulfinyls) such as methylsulfinyl, ethylsulfinyl, phenylsulfinyl and p-methylphenylsulfinyl; alkyl- and aryl-sulfonyls (preferably substituted or non-substituted C₁₋₃₀ alkylsulfonyls and substituted or non-substituted C₆₋₃₀ arylsulfonyls) such as methylsulfonyl, ethylsulfonyl, phenylsulfonyl and p-methylphenylsulfonyl; acyls (preferably formyl, substituted or non-substituted C₂₋₃₀ alkylcarbonyls and substituted or non-substituted C₇₋₃₀ arylcarbonyls) such as formyl, acetyl and pivaloyl benzoyl; aryloxycarbonyls (preferably substituted or non-substituted C₇₋₃₀ aryloxycarbonyls) such as phenoxycarbonyl, o-chlorophenoxycarbonyl, m-nitrophenoxy carbonyl and p-tert-butylphenoxy carbonyl; alkoxycarbonyls (preferably substituted or non-substituted C₂₋₃₀ alkoxycarbonyls) such as methoxycarbonyl, ethoxycarbonyl, tert-butoxycarbonyl and n-octadecyloxycarbonyl; carbamoyls (preferably substituted or non-substituted C₁₋₃₀ carbamoyls) such as non-substituted carbamoyl, N-methylcarbamoyl, N,N-dimethylcarbamoyl, N,N-di-n-octylcarbamoyl and N-(methylsulfonyl)carbamoyl; aryl- and heterocyclic azo groups (preferably substituted or non-substituted C₆₋₃₀ arylazo groups and substituted or non-substituted C₃₋₃₀ heterocyclic azo groups) such as phenylazo, p-chlorophenylazo and 5-ethylthio-1,3,4-thiaziazol-2-yl azo; imido groups such as N-succinimido and N-phthalimido; phosphinos (preferably substituted or non-substituted C₂₋₃₀ phosphinos) such as dimethyl phosphino, diphenyl phosphino and methylphenoxy phosphino; phosphinyls (preferably substituted or non-substituted C₂₋₃₀ phosphinyls) such as non-substituted phosphinyl, dioctyloxy phosphinyl and diethoxy phosphinyl; phosphinyloxys (preferably substituted or non-substituted C₂₋₃₀ phosphinyloxys) such as diphenoxy phosphinyloxy and dioctyloxy phosphinyloxy; phosphinylaminos (preferably substituted or non-substituted C₂₋₃₀ phosphinylaminos) such as dimethoxy phosphinylamino and dimethylamino phosphinylamino; and silyl groups (preferably substituted or non-substituted C₃₋₃₀ silyl groups) such as trimethyl silyl, tert-butyl dimethyl silyl and phenyldimethyl silyl.

The exemplified substituents having at least one hydrogen atom the exemplified substituents may have at least one substituent in place of hydrogen atom. Examples of such a substituent include alkylcarbonylaminosulfonyl, arylcarbonylaminosulfonyl, alkylsulfonylaminocarbonyl and arylsulfonylaminocarbonyl. Specific examples include methylsulfonylaminocarbonyl, p-methylphenylsulfonylaminocarbonyl, acetylaminosulfonyl and benzoyl aminosulfonyl.

R¹ is preferably a halogen atom, alkyl group, alkenyl group, aryl group, heterocyclic group, hydroxyl, carboxyl, alkoxy group, aryloxy group, acyloxy group, cyano or amino, and more preferably a halogen atom, alkyl group, cyano group or alkoxy group.

When there are a plurality of (R¹)s, and such (R¹)s form a ring, the ring may preferably be a five- to eight-membered ring, and more preferably a five- or six-membered ring.

In formula (I), “m” represents the number of substitution of R¹, and may vary depending on structures of the ring formed by Z together with C—C═C—C or C═C—C═C. In the formula, “m” may have the smallest value of 0, and the largest value of 4 for the case where Z represents two carbon atoms, and the ring formed by Z together with C—C═C—C or C═C—C═C is non-aromatic. Preferably, m is 0 or 1, and is more preferably 0.

In formula (i), each of R² and R³ independently represents a substituent. Examples of the substituent may be those listed for R¹ in the above. In order to allow the compound of the present invention to express a stronger reversed wavelength dispersion of Δn, each of R² and R³ preferably aligns in the longitudinal direction of the molecule. For this purpose, each substituent preferably has a certain length of substituent including a chain structure and/or cyclic structure. The compound represented by formula (I) also preferably expresses liquid crystallinity. As a factor for expressing liquid crystallinity, the molecule needs a rigid portion called core, and a flexible portion called side chain, as described in “Ekisho Binran (Handbook of Liquid Crystal)”, published by Maruzen Co., Ltd., 2000, Chapter 3 “Bunshi Kozo to Ekisho-sei (Molecular Structure and Liquid Crystallinity)”. Accordingly, each of the substituents R² and R³ preferably has at least one rigid portion, or a cyclic portion, and more preferably contains two or more cyclic structures. Additionally, selecting the same group for both of R²-L¹- and R³-L²- may be advantageous in terms of producing the compound at a low cost.

In formula (I), each of R² and R³ may preferably be a substituted or non-substituted phenyl group, or substituted or non-substituted cyclohexyl group. More preferably, it may be a substituted phenyl group, or a substituted cyclohexyl group, and still more preferably a phenyl group substituted at the 4-position, or a cyclohexyl group substituted at the 4-position. Further more preferably, it may be a phenyl group substituted at the 4-position thereof with a phenylene group substituted at the 4-position, bound directly or via a linking group; a phenyl group substituted at the 4-position thereof with a cyclohexyl group substituted at the 4-position, bound directly or via a linking group; a cyclohexyl group substituted at the 4-position thereof with a phenyl group substituted at the 4-position, bound directly or via a linking group; or a cyclohexyl group substituted at the 4-position thereof with a cyclohexyl group substituted at the 4-position, bound directly or via a linking group. More specifically, they are the groups shown below.

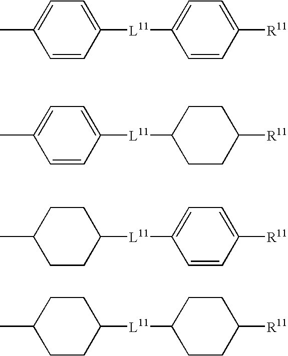

In the each formula, L¹¹ represents a single bond or linking group, and R¹¹ is a substituent. Examples of the linking group represented by L¹¹ are same as those exemplified as examples of L¹ and L² in the above. Among these, a single bond, oxycarbonyl group (—O—C(═O)—) and carbonyloxy group (—C(═O)O—) are preferable. Examples of the substituent represented by R¹¹ may be similar to those represented by R¹. Among these, substituted or non-substituted alkylcarbonyloxy group (including cycloalkylcarbonyloxy group) having 1 to 10 carbon atoms, substituted or non-substituted alkoxy group having 1 to 10 carbon atoms, and substituted or non-substituted arylcarbonyloxy group having 6 to 16 carbon atoms are preferable; and substituted or non-substituted alkylcarbonyloxy group having 1 to 8 carbon atoms, and substituted or non-substituted alkoxy group having 1 to 10 carbon atoms are more preferable. In the alkyl portion of the alkylcarbonyloxy group or alkoxy group, either one of the non-adjacent carbon atoms may be substituted by an oxygen atom, sulfur atom, —CH═CH— or —C≡C—. Also the groups shown below, the terminal of the alkyl portions of which being substituted by a polymerizable group described later, such as P3, are listed as preferable examples, without any limitation.

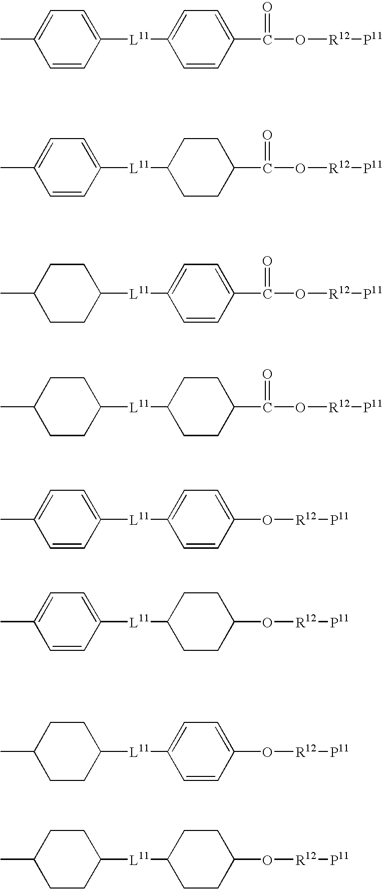

In the formulae, L¹¹ is similarly defined as described in the above. R¹² represents an alkyl group (either one of non-adjacent carbon atoms of which may be substituted by an oxygen atom or sulfur atom, and is preferably an alkyl group of C₁ to C₁₀ or around). P¹¹ represents a polymerizable group, wherein specific examples of which include P1 to P4 shown later.

The cyclohexyl group substituted at the 4-position include streomers having cis form and trans form. The present invention is not limited to either of them, instead allowing both of cis form and trans form, and even mixtures of the both. Trans-cyclohexyl group is more preferable.

In formula (I), each of A¹ and A² independently represents a group selected from the group consisting of —O—, —NR— (R represents hydrogen atom or substituent), —S— and —CO—, preferably represents —O—, —NR— (R represents a substituent, examples of which may be those exemplified for R¹ in the above.) or —S—.

Each of B¹ and B² independently represents a group selected from the group consisting of —O—, —S—, —SO—, —SO₂—, —NR— (R represents a hydrogen atom or substituent, examples of which may be those exemplified for R¹ in the above.), ═N—, ═N⁽⁺⁾R— (R represents a hydrogen atom or substituent, examples of which may be those exemplified for R¹ in the above.), —CO—, —CS—, and ═CR— (R represents a hydrogen atom or substituent, examples of which may be similar to those exemplified for R¹ in the above.). Y represents two to four atoms selected from the group consisting of carbon atom and the Group XIV to Group XVI non-metallic atoms, and forms a five- to seven-membered ring together with B¹—C—B² illustrated in the formula. If the atom(s) embedded in a ring Y has substitutable hydrogen atom(s) bonded thereto, such hydrogen atom(s) may be substituted by a substituent R⁴, and examples of R⁴ include those exemplified above as examples of R¹. The ring formed by Y and B¹—C—B² may preferably be a five- or six-membered ring. Specific examples of preferable cyclic structure will be shown below. In the structural formulae, “*” indicates a site of linking with the five-membered ring containing A¹ and A².

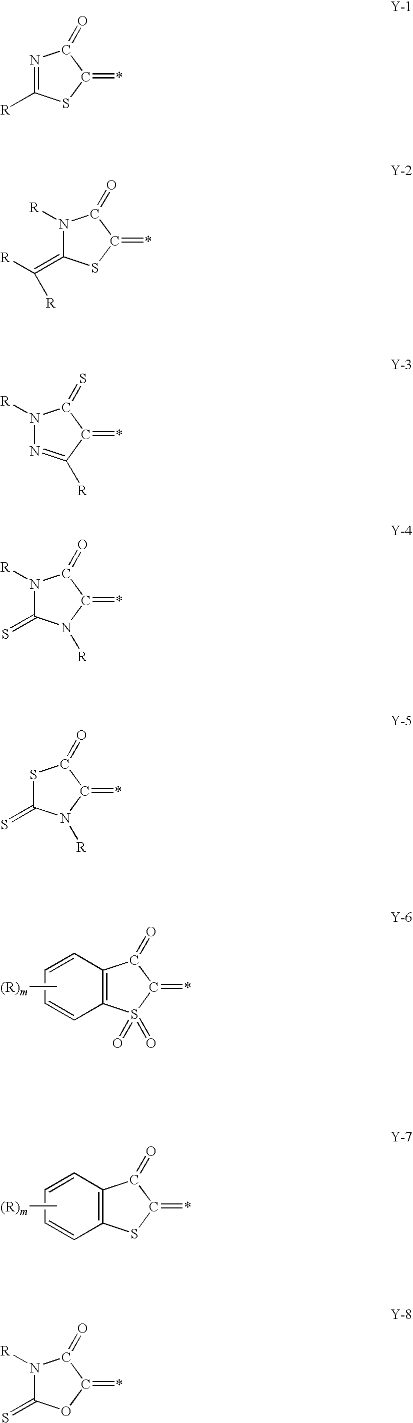

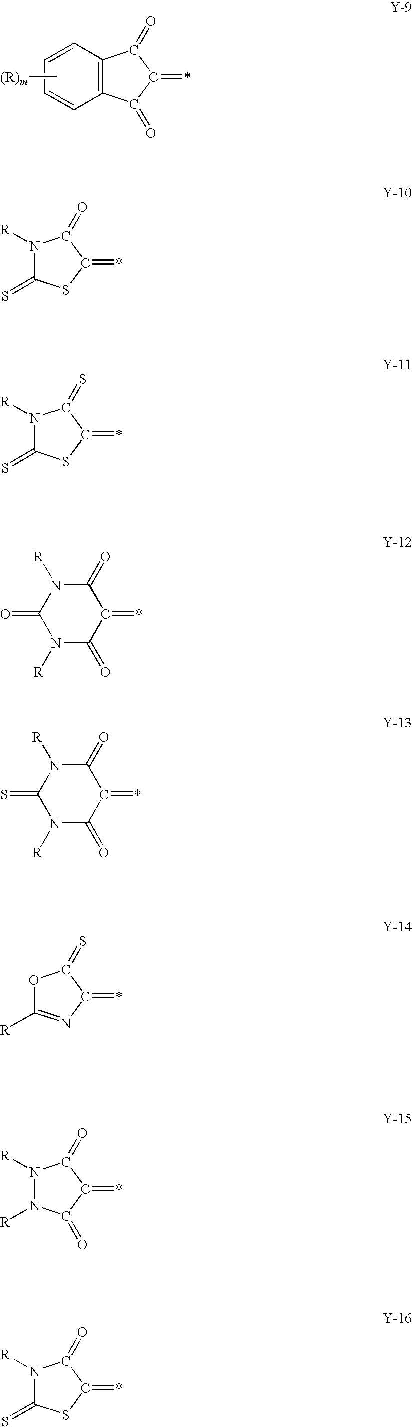

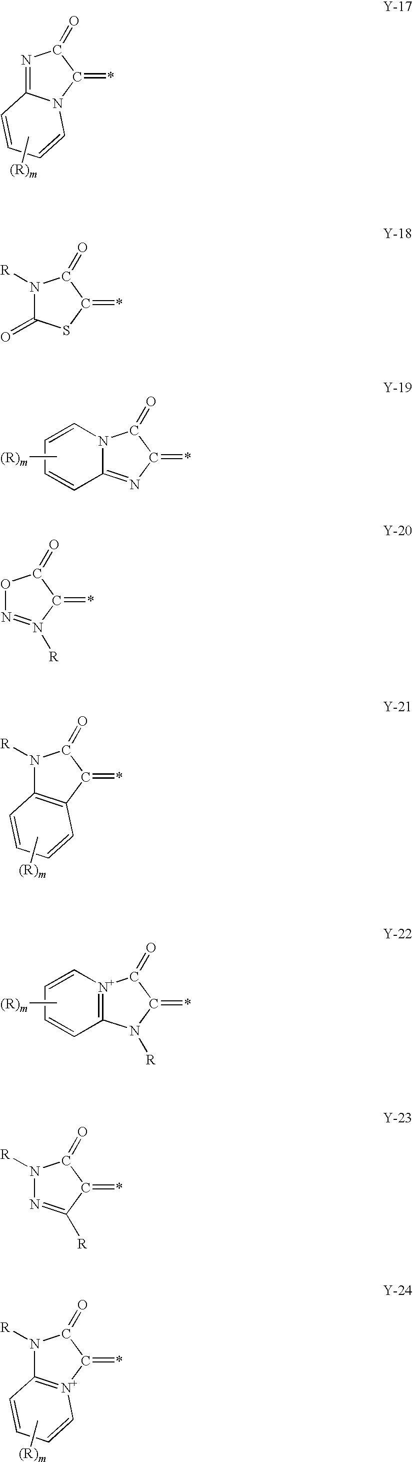

In the formulae, each R represents same or different substituent or a hydrogen atom. In the formulae, m represents an integer from 0 to 4. Examples of the substituent include those exemplified above as examples of R¹. As an example of R in Y-1, —N (Ph)₂ (where, Ph is a phenyl group) may be exemplified. Examples of R bound to N in Y-2 include alkyl groups of C₁₋₃ or around, such as ethyl group, and examples of two (R)s bound to C═ include CN. Examples of R substituted on N in Y-3 include substituted or non-substituted phenyl groups, and examples of R bound to C include alkyl groups of C₁₋₃ or around, such as methyl group. Examples of R bound to each of two (N)s in Y-4 include alkyl groups of C₁₋₃ or around, such as ethyl group, and substituted or non-substituted phenyl groups. Examples of R bound to N in each of Y-5 and Y-8 include alkyl groups of C₁₋₃ or around, such as ethyl group. Examples of R bound to N in Y-10 include substituted or non-substituted phenyl groups. Examples of R(s) bound to N(s) in each of Y-11, Y-12 and Y-13 include alkyl groups of C₁₋₃ or around, such as ethyl group. Examples of R bound to N in Y-14 include substituted or non-substituted phenyl groups. Examples of (R)s bound to each of two (N)s in Y-15 include alkyl groups of C₁₋₃ or around, such as methyl group, and substituted or non-substituted phenyl groups. Examples of R bound to N in Y-16 include alkyl groups of C₁₋₃ or around, such as ethyl group. Examples of R bound to N of each of Y-18 and Y-20 include substituted or non-substituted phenyl groups. Examples of R bound to N in each of Y-21 and Y-22 include alkyl groups of C₁₋₃ or around, such as methyl group and ethyl group. Examples of R bound to N in Y-23 include substituted or non-substituted phenyl groups, and examples of R bound to C include hydrogen atom, OH and ester group (COOR: R represents alkyl groups of C₁₋₃ or around, such as ethyl group). Examples of R bound to N in Y-24 include alkyl groups of C₁₋₃ or around, such as methyl group.

Among these examples, Y-1, Y-3, Y-5, Y-8, Y-9, Y-12, Y-13, Y-15, Y-21 and Y-23 are preferable as ring Y. Particularly preferable examples include Y-5, Y-9, Y-13, Y-15 and Y-23.

Examples of the compound represented by the formula (I) include those represented by formula (I)′ below, having —S— for A¹ and A².

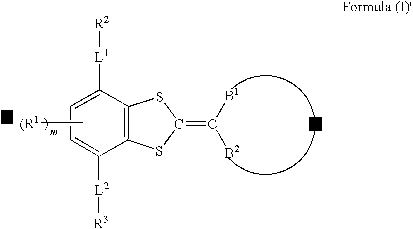

In formula (I)′, S represents a sulfur atom. Any other symbols, and also preferable ranges thereof, are defined similarly to those in formula (I).

Examples of the compound represented by formula (I) include those represented by formulae (I-a) to (I-e), having —S— for A¹ and A², and having any one of Y-9, Y-13, Y-15, Y-23 and Y-5 for ring Y

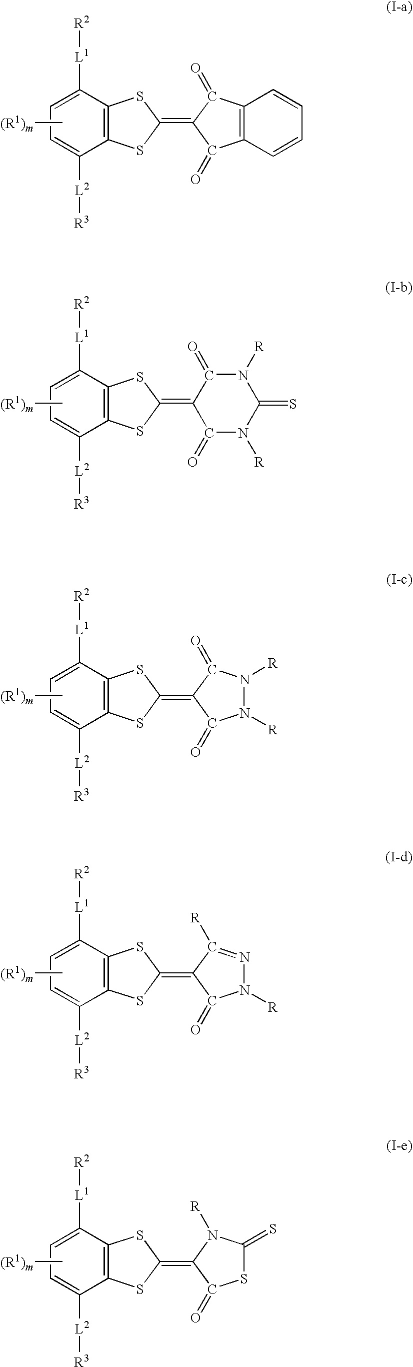

In formulae (I-a) to (I-e), S represents a sulfur atom. Any other symbols, and also preferable ranges thereof, are defined similarly to those in the formula (I), and those in Y-9, Y-13, Y-15, Y-23 and Y-5 shown in the above.

As for a reason why the compounds (in particular those represented by formulae (I-a) to (I-e)) of the present invention shows reversed wavelength dispersion characteristics of Δn, the present inventor supposes that each compound of the present invention has a cyclic structure in which the aromatic ring thereof, substituted with R²-L¹ and R³-L², develops larger dipole moment in the lateral direction. The molecules of the compounds of the present invention may align in a liquid crystal layer in the manner that the long molecular axes composed of R²-L¹, aromatic ring and R³-L² uniformly align, which shows refractive index anisotropy (Δn). In such an alignment state, the dipole moment may be developed in the lateral direction of the long molecular axis; and, as a result, the refractive index anisotropy (Δn) may show reversed wavelength dispersion characteristics.

The compound of the present invention may also have polymerizable group(s). Optical components, such as retardation plate, manufactured by using the compound having polymerizable group(s), may successfully be prevented from causing variation in the optical characteristics (retardation) due to heat and so forth. The polymerizable group(s) is preferably located at terminal(s) of molecule of the compound. From this point of view, at least one of R, R¹, R², R³, and R⁴ in formula (I) preferably has a polymerizable group on the terminal(s) thereof. The compound preferably has 1 to 6, more preferably 1 to 4, and still more preferably 2 or 3 polymerizable groups in each molecule. In particular, at least one of R², R³ and R⁴ preferably has a polymerizable group, at least either one of R² and R³ more preferably has a polymerizable group, and both of R² and R³ still more preferably have the polymerizable groups.

The number of the polymerizable groups in the compound of the present invention is not specifically limited. Preferable examples of the polymerizable group include groups capable of proceeding addition polymerization reaction or condensation polymerization reaction. Polymerizable ethylenic unsaturated group or ring-opening polymerizable group is preferable as the polymerizable groups. Examples of the polymerizable group will be shown below.

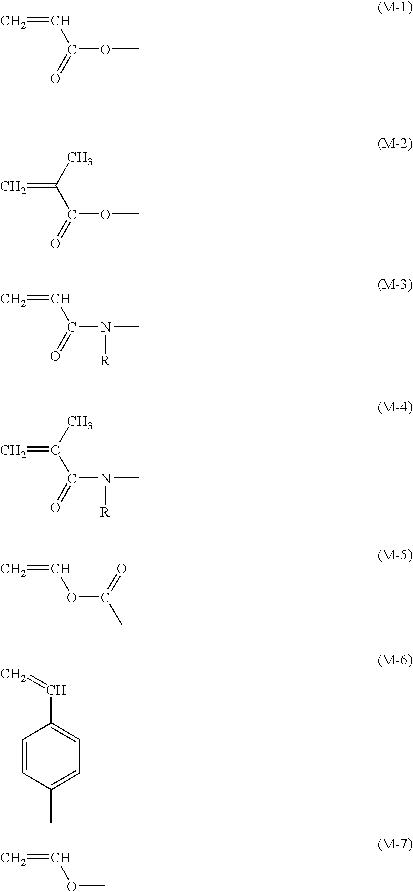

Furthermore, the polymerizable group is particularly preferably a functional group capable of proceeding addition polymerization reaction. This sort of polymerizable group is preferably a polymerizable ethylenic unsaturated group or ring-opening polymerizable group.

The polymerizable group is preferably a group represented by any of the formulae P1, P2, P3 and below.

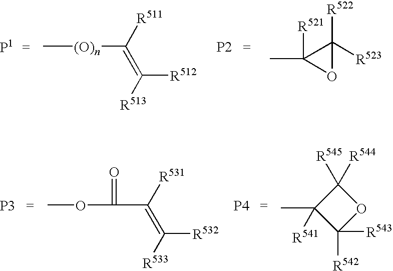

In the formulae, each of R⁵¹¹, R⁵¹², R⁵¹³, R⁵²¹, R⁵²², R⁵²³, R⁵³¹, R⁵³², R⁵³³, R⁵⁴¹, R⁵⁴², R⁵⁴³, R⁵⁴⁴ and R⁵⁴⁵ independently represents a hydrogen atom or alkyl group. n is 0 or 1.

Each of R⁵¹¹ R⁵¹² and R⁵¹³ in polymerizable group P1 independently represents a hydrogen atom or alkyl group.

The polymerizable group P1 may be bound, for example, to an alkoxy group, alkoxycarbonyl group and alkoxycarbonyloxy group residues. Examples of the alkoxy group, alkoxycarbonyl group and alkoxycarbonyloxy group residues include alkyleneoxy group (for example, alkyleneoxy groups such as ethyleneoxy, propyleneoxy, butyleneoxy, pentyleneoxy, hexyleneoxy, and heptyleneoxy groups; and substituted alkyleneoxy groups having ether bond, such as ethyleneoxyethoxy group); alkyleneoxycarbonyloxy group (for example, alkyleneoxycarbonyloxy groups such as ethyleneoxycarbonyloxy, propyleneoxycarbonyloxy, butyleneoxycarbonyloxy, pentyleneoxycarbonyloxy, hexyleneoxycarbonyloxy, and heptyleneoxycarbonyloxy groups; and substituted alkyleneoxycarbonyloxy groups having ether bond, such as ethyleneoxyethoxycarbonyloxy group); and alkyleneoxycarbonyl group (for example, alkyleneoxycarbonyl groups such as ethyleneoxycarbonyl, propyleneoxycarbonyl, butyleneoxycarbonyl, pentyleneoxycarbonyl, hexyleneoxycarbonyl and heptyleneoxycarbonyl groups; and substituted alkyleneoxycarbonyl groups having ether bond, such as ethyleneoxyethoxycarbonyl group). The polymerizable group P1 may directly be bound to atoms(s) composing L¹ and L², and the rings Z and Y.

In the formula, “n” represents an integer of 0 or 1, preferably represents 1. When “n” is 1, the polymerizable group P1 represents a substituted or non-substituted vinyl ether group. Each of R⁵¹¹ and R⁵¹³ independently represents a hydrogen atom, or alkyl group (for example, methyl, ethyl, n-propyl, isopropyl, n-butyl, pentyl, hexyl, heptyl, octyl and nonyl, wherein lower alkyl groups such as methyl and ethyl are preferable, and methyl group is more preferable). A combination having a methyl group for R⁵¹¹ and a hydrogen atom for R⁵¹³, or a combination having hydrogen atoms both for R⁵¹¹ and R⁵¹³, is preferable.

In the formula, R⁵¹² represents a hydrogen atom, or substituted or non-substituted alkyl group (for example, methyl, ethyl, n-propyl, isopropyl, n-butyl, pentyl, hexyl, heptyl, octyl, nonyl, 2-chloroethyl, 3-methoxyethyl and methoxyethoxyethyl groups are exemplified, wherein lower alkyl groups such as methyl and ethyl groups are preferable, and methyl group is more preferable), wherein hydrogen atom and lower alkyl groups are preferable, and hydrogen atom is more preferable. Accordingly, as the polymerizable group P1, a non-substituted vinyloxy group, which is a functional group having a high polymerization activity, may generally be preferable.

The polymerizable group P2 represents a substituted or non-substituted oxylane group. Each of R⁵²¹ and R⁵²² independently represents a hydrogen atom, alkyl group (for example, methyl, ethyl, n-propyl, isopropyl, n-butyl, pentyl, hexyl, heptyl, octyl and nonyl are exemplified, wherein lower alkyl groups such as methyl and ethyl are preferable, and methyl is more preferable). Both of R⁵²¹ and R⁵²² preferably have hydrogen atoms.

In the formula, R⁵²³ represents a hydrogen atom, substituted or non-substituted alkyl group (for example, methyl, ethyl, n-propyl, isopropyl, n-butyl, pentyl, hexyl, heptyl, octyl, nonyl, 2-chloroethyl, 3-methoxyethyl and methoxyethoxyethyl are exemplified, wherein lower alkyl groups such as methyl and ethyl are preferable, and methyl is more preferable), wherein hydrogen atom, or lower alkyl groups such as methyl, ethyl and n-propyl are preferable.

The polymerizable group P3 represents a substituted or non-substituted acryl group. Each of the substituent R⁵³¹ and R⁵³³ independently represents a hydrogen atom, or alkyl group (for example, methyl, ethyl, n-propyl, isopropyl, n-butyl, pentyl, hexyl, heptyl, octyl and nonyl are exemplified, wherein lower alkyl groups such as methyl and ethyl are preferable, and methyl is more preferable). A combination having a methyl group for R⁵³¹ and a hydrogen atom for R⁵³³, or a combination having hydrogen atoms both for R⁵³¹ and R⁵³³, is preferable.

In the formula, R⁵³² represents a hydrogen atom, or substituted or non-substituted alkyl group (for example, methyl, ethyl, n-propyl, isopropyl, n-butyl, pentyl, hexyl, heptyl, octyl, nonyl, 2-chloroethyl, 3-methoxyethyl and methoxyethoxyethyl are exemplified, wherein lower alkyl groups such as methyl and ethyl are preferable, and methyl is more preferable), wherein hydrogen atom is preferable. Accordingly, as the polymerizable group P3, functional groups having high polymerization activities, such as non-substituted acryloxy group, methacryloxy group, crotonyloxy, are generally be preferable.

The polymerizable group P4 represents a substituted or non-substituted oxetane group. Each of R⁵⁴², R⁵⁴³, R⁵⁴⁴ and R⁵⁴⁵ independently represents a hydrogen atom, alkyl group (for example, methyl, ethyl, n-propyl, isopropyl, n-butyl, pentyl, hexyl, heptyl, octyl and nonyl are exemplified, wherein lower alkyl groups such as methyl and ethyl are preferable, and methyl is more preferable). All of R⁵⁴², R⁵⁴³, R⁵⁴⁴ and R⁵⁴⁵ are preferably hydrogen atoms.

In the formula, R⁵⁴¹ represents a hydrogen atom, substituted or non-substituted alkyl group (for example, methyl, ethyl, n-propyl, isopropyl, n-butyl, pentyl, hexyl, heptyl, octyl, nonyl, 2-chloroethyl, 3-methoxyethyl and methoxyethoxyethyl are exemplified, wherein lower alkyl groups such as methyl and ethyl are preferable, and methyl is more preferable), wherein hydrogen atom, or lower alkyl groups such as methyl, ethyl and n-propyl are preferable.

Specific examples of the compound represented by formula (I) include, but are not limited to, those shown below.

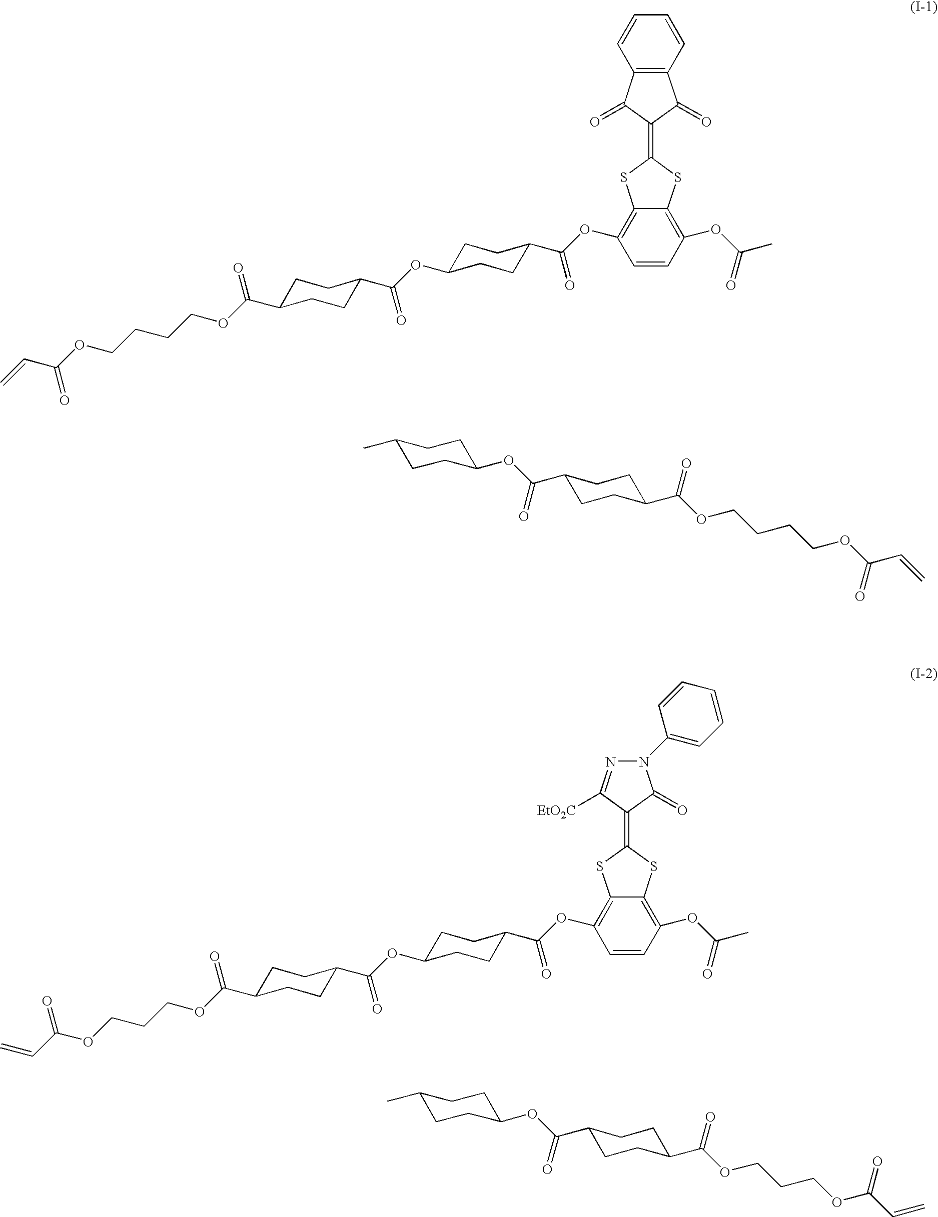

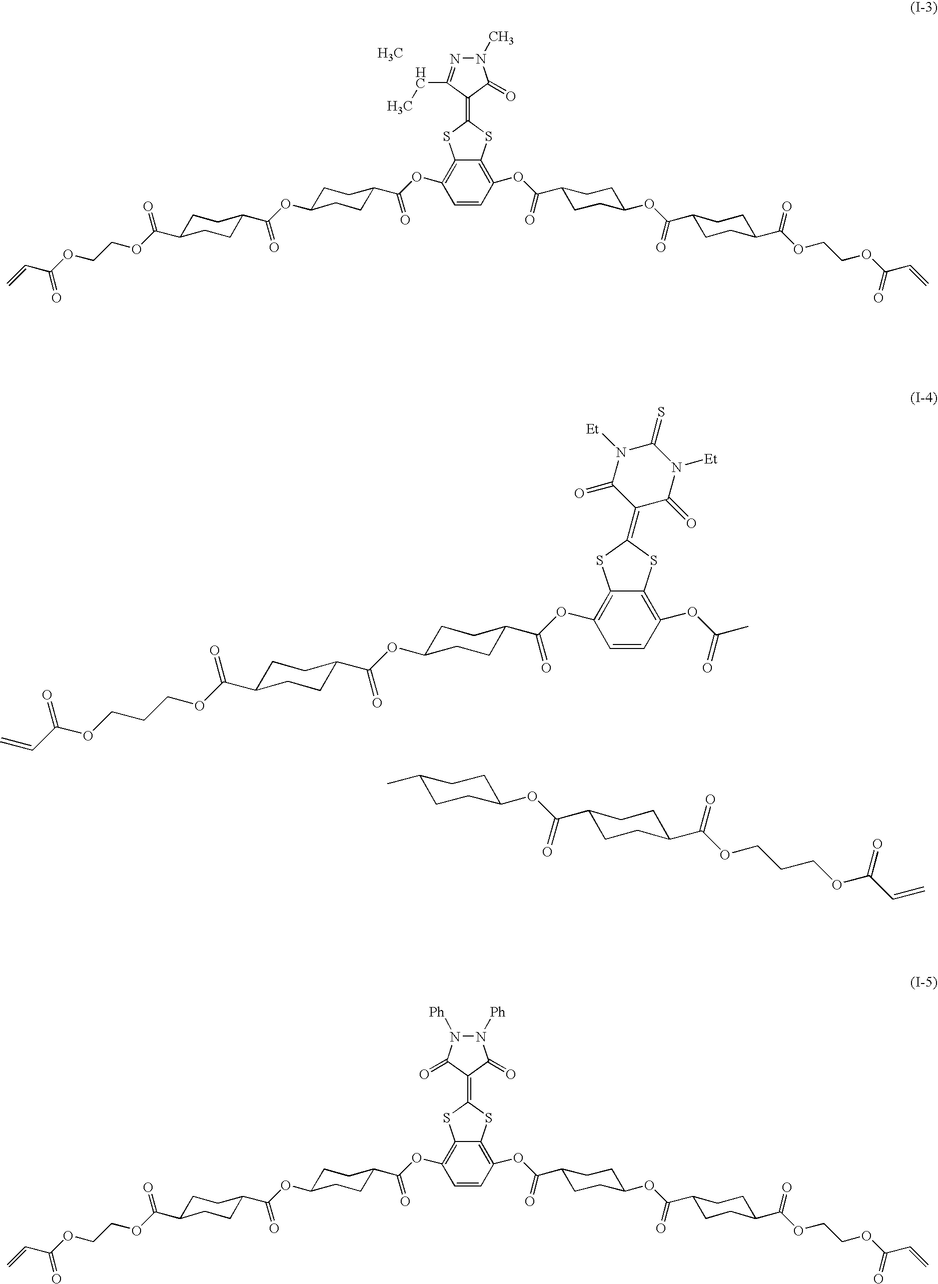

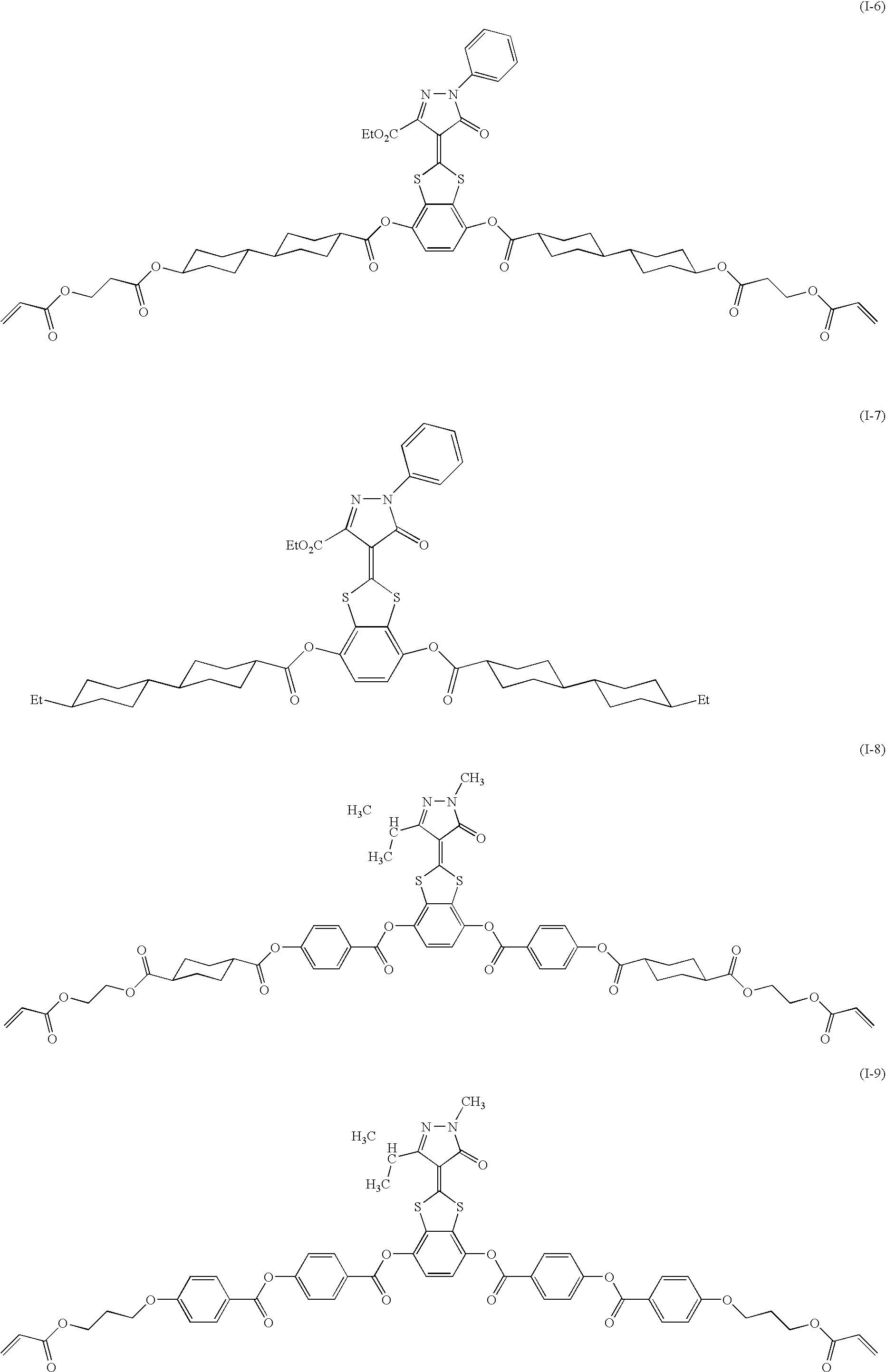

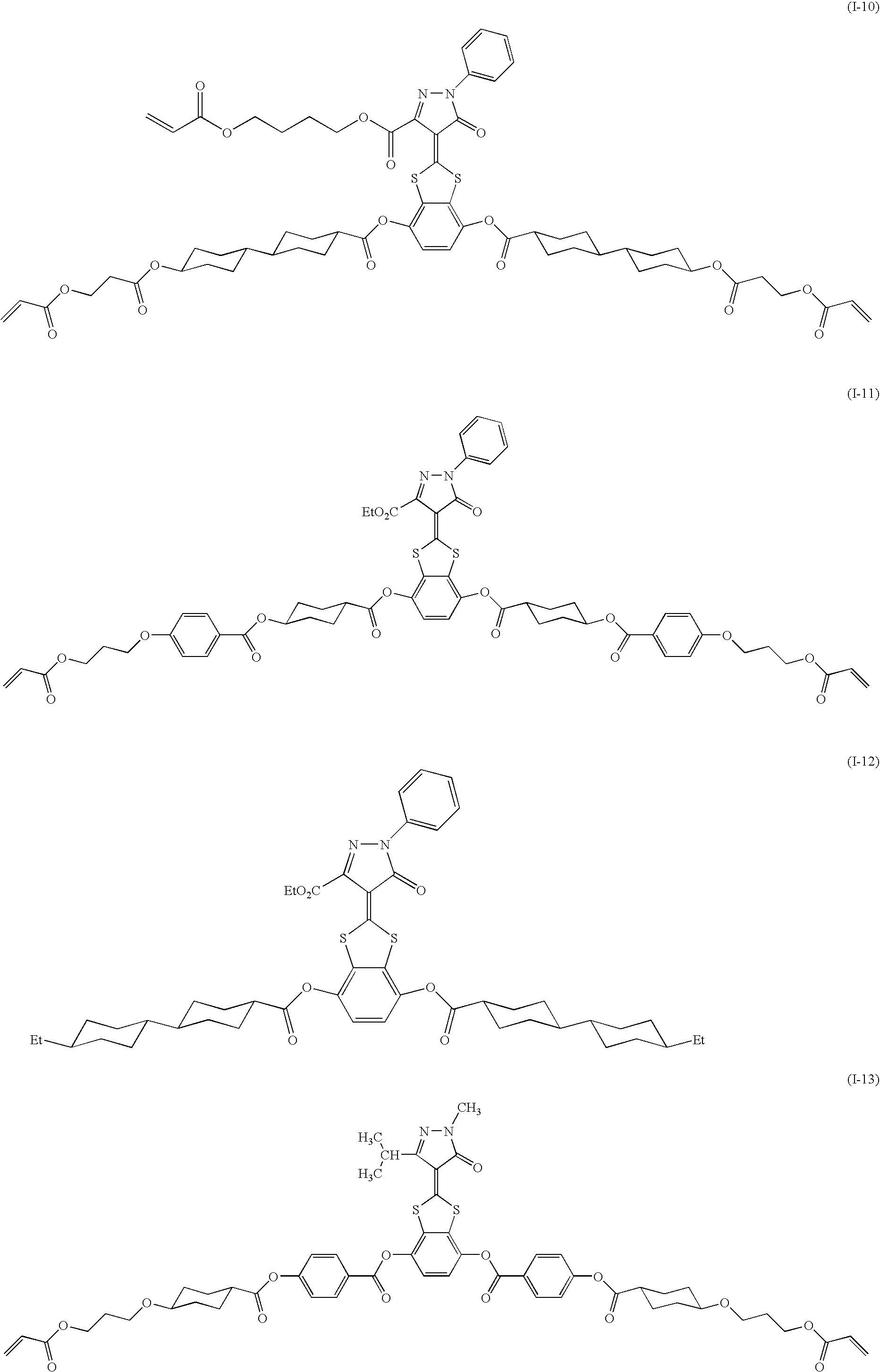

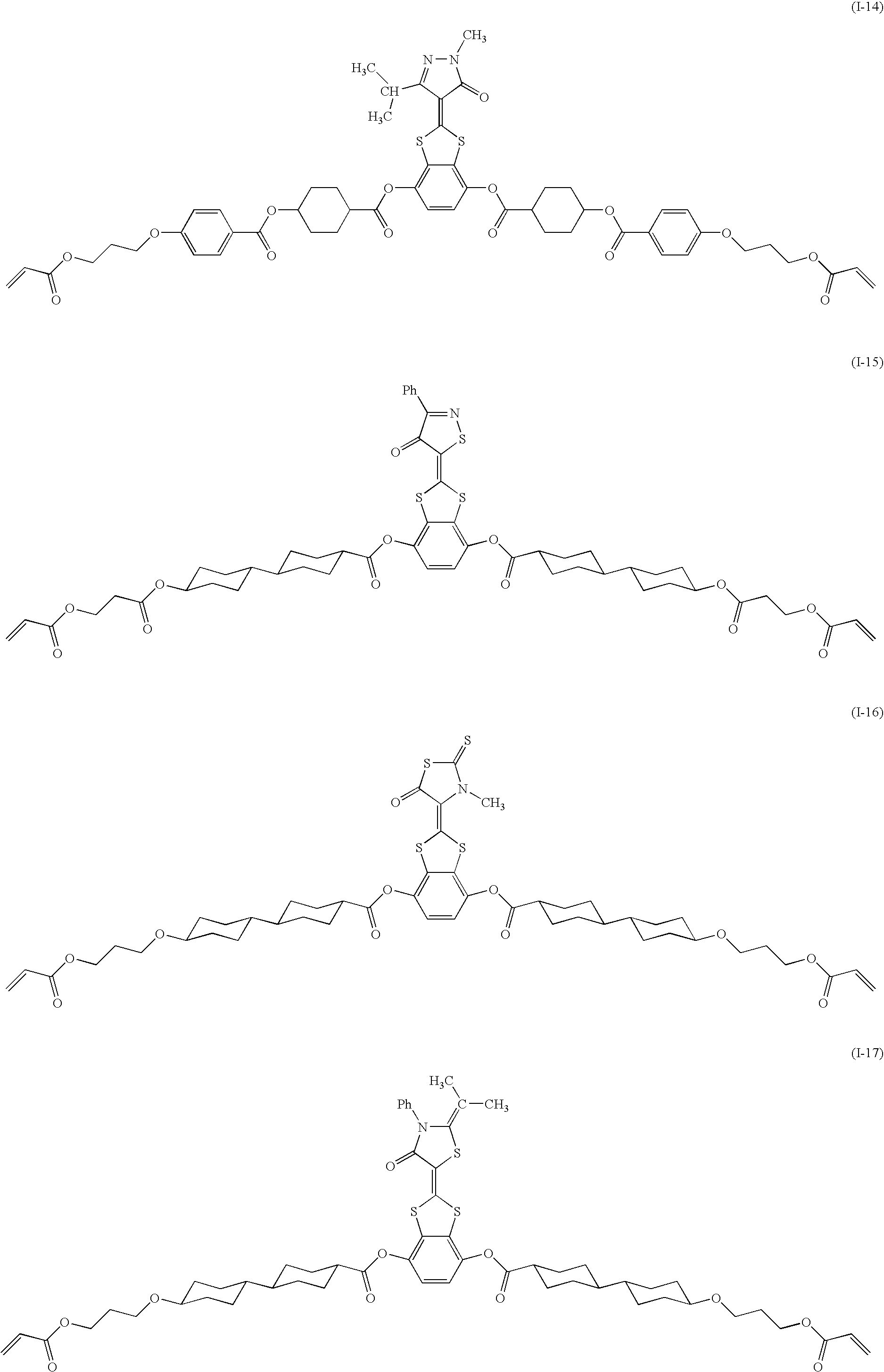

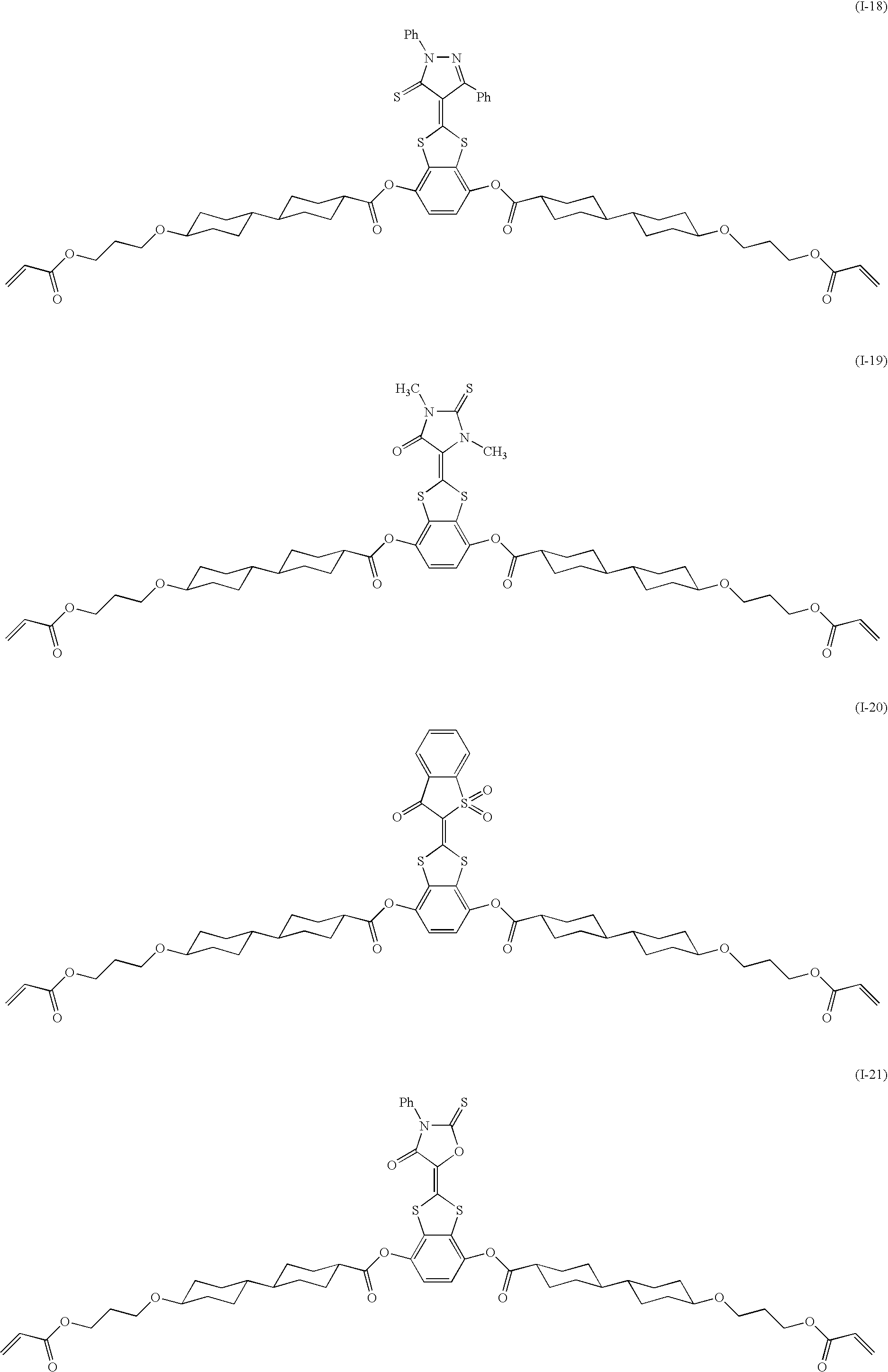

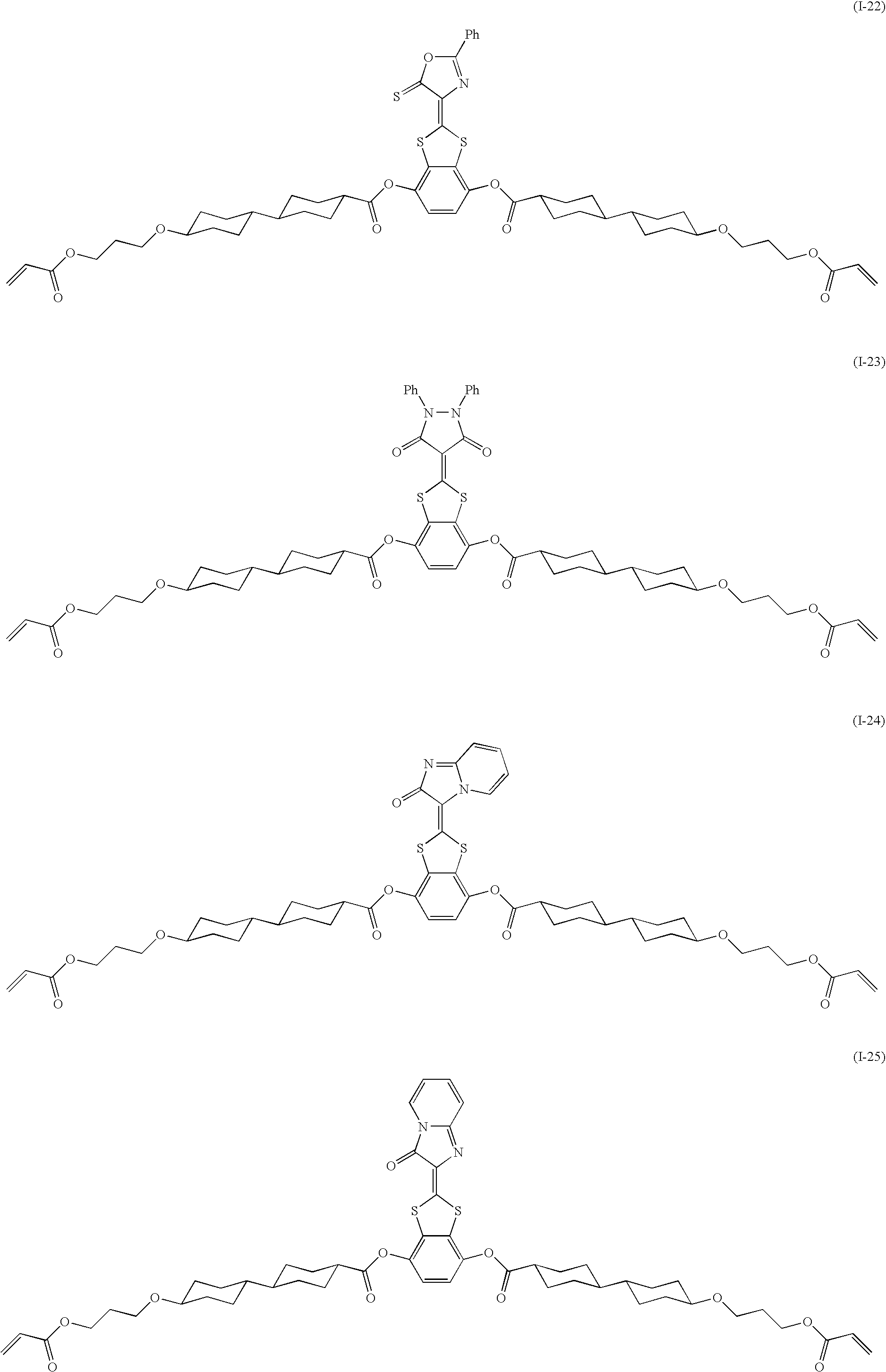

Although the examples enumerated in the above were those having the polymerizable groups P3 bonded to the terminals of R² and R³, examples of the present invention also include compounds having any of the polymerizable groups P1, P2 and P4, in place of the polymerizable groups P3. Of course, examples of the invention also include compounds having no polymerizable groups bound to the terminals of R² and R³, such as those replacing the polymerizable groups P3 with substituents such as hydrogen atoms, halogen atoms and so forth.

The wavelength dispersion characteristics of Δn of the compound of the present invention cannot unequivocally be limited, because preferable ranges thereof may vary depending on its applications. For the purpose of producing a wide λ/4 plate, Δn preferably satisfies conditions (4) and (5) below:

0.60<Δn(450 nm)/Δn(550 nm)<0.99 (4)

1.01<Δn(650 nm)/Δn(550 nm)<1.35 (5)

Examples of the compound of the invention include both of the compounds showing positive and negative birefringence; and the compounds showing positive birefringence are more preferable.

Liquid crystal phase showing positive birefringence is detailed, for example, in “Ekisho Binran (Handbook of Liquid Crystal)”, published by Maruzen Co., Ltd., 2000, Chapter 2, and may be exemplified by nematic phase, cholesteric phase, and smectic phase (for example, smectic-A phase and smectic-C phase).

For using the compound of the present invention to prepare an optically anisotropic layer, the compound preferably shows a desirable mono-domain property in view of obtaining a uniform and defect-less alignment. Undesirable mono-domain property may makes the resultant structure poly-domain-like, which is causative of scattering of light due to alignment failure possibly occurs at the boundary between the adjacent domains. This is undesirable also from the viewpoint of degradation in the transmittance of the optically anisotropic layer. For the purpose of expressing a desirable mono-domain property, the compound of the present invention preferably expresses nematic phase (N phase) or smectic-A phase (SA phase). It is particularly preferable to express nematic phase.

Examples of the compound of the invention include both of low-molecular-weight compounds and high-molecular-weight compounds; and the low-molecular-compounds are more preferable in terms of alignment of the liquid crystal.

### [Optically Anisotropic Material]

The present invention relates also to an optically anisotropic material composed of the liquid crystalline composition containing the compound of the present invention. One embodiment of the optically anisotropic material relates to an optically anisotropic film.

The optically anisotropic film of the present invention may be prepared as follows. A liquid crystal composition containing at least one species of the compound of the present invention is applied to a surface (preferably a surface of an alignment film), and aligned in any alignment state; and then fixing of the alignment is carried out. The compound used in such a process preferably expresses a nematic phase or smectic phase by itself, or under the presence of other additive(s).

The compound of the present invention may be used alone, or in combination with a plurality of other polymerizable compounds. Also any combination with a non-polymerizable compound is usable. Each compound used alone by itself or in combination may form nematic liquid crystal phase, smectic liquid crystal phase, or cholesteric liquid crystal phase, and preferably expresses the nematic phase, smectic phase or cholesteric phase within a temperature range capable of allowing the liquid crystal composition containing additive(s), aimed at forming the optically anisotropic film of the present invention, to align and fix (typically in a state having a solvent vaporized off in the process of drying under heating, if the composition is given in a form of coating liquid containing the solvent).

### Additive

The composition to be used for preparing the optically anisotropic film of the invention may contain an additive capable of promoting the orientation of molecules of the compound of the invention. The amount of the additive in the composition is preferably from 0.01 to 10% by mass, more preferably from 0.05 to 5% by mass and even more preferably from 0.05 to 4% by mass with respect to the amount of the compound of the invention (according to the embodiment employing other liquid crystal compound(s) along with the compound of the invention, the total amount of them). The additive capable of promoting orientation may contribute to aligning molecules of the compound of the invention at the air-interface or the alignment-layer interface with an excluded volume effect or electrostatic effect. The compounds disclosed in JPA Nos. 2002-20363 and 2002-129162 may be used Further, the matters disclosed in JPA No. 2004-53981, [0072]-[0075], JPA No. 2004-4688, [0071]-[0078], or JPA No. 2004-139015, [0052]-[0054], [0065]˜[0066] and [0092]-[0094] may be also applied to the invention.

### Chain Transfer Agent

The composition to be used for preparing the optically anisotropic film of the invention may contain a chain transfer agent. The amount of the chain transfer agent in the composition is preferably from 0.01 to 10% by mass, more preferably from 0.05 to 5% by mass and even more preferably from 0.05 to 4% by mass with respect to the amount of the compound of the invention (according to the embodiment employing other liquid crystal compound(s) along with the compound of the invention, the total amount of them). Any known normal chain transfer agents may be used; and preferable examples of the chain transfer agent include compounds having a mercapto group such as thiol compounds (including dodecyl mercaptan, octyl mercaptan, trimethylol propane tris(3-mercapto propionate) and pentaerythritol tetrakis(3-mercapto propionate), etc.) and disulfide compounds (including diphenyl disulfide, etc.). The chain transfer agent may be required to have compatibility with the compound of the invention and other compound(s) to be added optionally thereto; and in terms of compatibility, thiol compounds exhibiting liquid-crystallinity are preferable. Examples of the thiol compound exhibiting liquid-crystallinity include compounds disclosed in U.S. Pat. No. 6,096,241.

### Other Materials in Composition for Preparing Optically Anisotropic Film

The composition, which may be a coating fluid, to be used for preparing the optically anisotropic film of the invention may contain the compound of the invention, and other liquid-crystal compound(s), the agent(s) capable for promoting orientation and the chain transfer agent (s) which are optionally added thereto; and the composition may further contain other material such as a polymerization initiator, plasticizer, surfactant and polymerizable monomer. The other materials may be used in terms of various purposes such as fixing alignment, improving uniformity of the coating layer, increasing film strength and enhancing alignment-ability of the compound of the invention or the optionally added liquid-crystal compound. It is preferable that the other materials show compatibility with the compound of the invention or the optionally added other liquid-crystal compound and don't prevent them from aligning in a preferred alignment.

The polymerization initiator may be selected from thermal polymerization initiators or photo-polymerization initiators. Photo-polymerization initiators are more preferable. Examples of the photo-polymerization initiator include α-carbonyl compounds (those described in U.S. Pat. Nos. 2,367,661 and 2,367,670), acyloin ethers (those described in U.S. Pat. No. 2,448,828), α-hydrocarbon-substituted aromatic acyloin compounds (those described in U.S. Pat. No. 2,722,512), polynuclear quinone compounds (those described in U.S. Pat. Nos. 3,046,127 and 2,951,758), combinations of triarylimidazole dimer and p-aminophenyl ketone (those described in U.S. Pat. No. 3,549,367), acrydine and phenazine compounds (those described in Japanese Unexamined Patent Publication JPA No. Syouwa 60-105667 and U.S. Pat. No. 4,239,850), and oxadiazole compounds (those described in U.S. Pat. No. 4,212,970).

The amount of the polymerization initiator in the composition is preferably from 0.01 to 20 mass %, and more preferably from 0.5 to 10 mass % with respect to the total mass of the composition (the total mass of the solid content when the composition is a coating fluid).

The polymerizable monomer may be selected from compounds capable of proceeding radial polymerization or compounds capable of proceeding cationic polymerization. Multifunctional monomers capable of proceeding radial polymerization are preferable; and, among those, the monomers capable of proceeding copolymerization with the above mentioned compound of the invention having a polymerizable group. Exampled of such a monomer include those described in JPA No. 2002-296423, [0018]-[0020]. The amount of the monomer maybe generally from about 1 to 50% by mass and is preferably from about 1 to 30% by mass with respect to the amount of the compound of the invention (according to the embodiment employing other liquid crystal compound(s) along with the compound of the invention, the total amount of them).

The surfactant may be selected from any known surfactants. Fluorochemical surfactants are preferable. Examples of the surfactant include those described in JPA No. 2001-330725, [0028]-[0056].

Polymer may be used along with the compound of the invention, and is preferably capable of improving viscosity of the coating fluid. Examples of such polymer include cellulose esters. Preferable examples of the cellulose ester include those described in JPA No. 2000-155216, [0178]. For avoiding disorder of alignment of the compound of the invention (according to the embodiment employing other liquid crystal compound(s) along with the compound of the invention, alignment of both of them), the amount of the polymer is preferably from about 0.1 to 10% by mass and more preferably from about 0.1 to 8% by mass with respect to the amount of the compound of the invention (according to the embodiment employing other liquid crystal compound(s) along with the compound of the invention, the total amount of them).

### Solvent

The composition may be prepared as a coating fluid. The solvent to be used for preparing the coating fluid is preferably selected from organic solvents. Examples of the organic solvent include amides such as N,N-dimethylformamide; sulfoxides such as dimethylsulfoxide; heterocyclic compounds such as pyridine; hydrocarbons such as benzene and hexane; alkyl halides such as chloroform and dichloromethane; esters such as methyl acetate and butyl acetate; ketones such as acetone and methyl ethyl ketone; and ethers such as tetrahydrofuran and 1,2-dimethoxyethane. Alkyl halides, esters or ketones are preferable; and esters or ketones are more preferable. Two or more species of organic solvents may be used in combination.

The coating fluid maybe applied to the surface according to any known method such as an extrusion coating method, direct gravure coating method, reverse gravure coating method, and die coating method.

According to the invention, the film may be prepared as follows. The coating fluid is applied to a surface of the alignment layer or the like; molecules of the compound of the invention are aligned in a desired alignment state; and then the alignment state is fixed. Fixing the alignment state may be achieved by carrying out polymerization of the polymerizable compound(s). Polymerization for fixing the alignment state is preferably carried out according to photo-polymerization process employing a photo-polymerization initiator. UV rays are preferably used for the photoirradiation. The irradiation energy is preferably from 20 mJ/cm² to 50 J/cm², more preferably from 100 to 800 mJ/cm². For promoting the photopolymerization reaction, the photoirradiation may be effected under heat.

The thickness of the optically anisotropic film is preferably from 0.1 to 10 μm, more preferably from 0.2 to 5 μm, and even more preferably from 0.5 to 5 μm.

We studied uniformity of the alignment prior to carrying out polymerization, and then found that preferred is that, after the coating fluid is applied to a surface and then a nematic phase or isotropic phase is obtained once, the phase is changed to a smectic phase by cooling and then the smectic phase is fixed. For obtaining a nematic phase or isotropic phase once, the temperature may be kept more than the smectic-phase transition temperature, Tsc. More specifically, the temperature is preferably kept at about Tsc+0.1° C. or higher, more preferably at about Tsc+1° C. or higher, and even more preferably at from Tsc+5° C. to Tsc+20° C. The heating treatment of the nematic or isotropic phase is preferably carried out according to the manner that the temperature of the phase is kept for a while, preferably for 10 seconds or more, more preferably for about 20 second or more, and even more preferably for about 30 seconds to 3 minutes.

### Alignment Layer

For preparing the optically anisotropic film of the invention, an alignment layer is preferably used. Generally, an alignment layer is capable of controlling the directions of liquid crystalline molecules. Furthermore, an alignment layer may be used for improving uniformity of alignment or enhancing adhesion between the optically anisotropic film and a polymer substrate which is used as a support of the film. It is to be noted that an alignment layer is removable after the alignment state has been fixed and the alignment layer has played its role. Or in other words, the fixed optically anisotropic layer may be transferred from on the alignment layer to on other support, a polarizing film or the like.

The alignment layer that can be employed in the present invention may be provided by rubbing a layer formed of an organic compound (preferably a polymer), oblique vapor deposition, the formation of a layer with microgrooves, or the deposition of organic compounds (for example, omega-tricosanoic acid, dioctadecylmethylammonium chloride, and methyl stearate) by the Langmuir-Blodgett (LB) film method. Further, alignment layers imparted with orientation functions by exposure to an electric or magnetic field or irradiation with light are also known.

The alignment layers formed by rubbing polymer layers are particularly desirable. The polymers used for preparing the alignment layers may basically have a molecular structure capable of aligning liquid-crystalline molecules. According to the present invention, the polymer is desirably selected from polymers having such a molecular structure and further having a structural feature in which a main chain bounds to side chains containing a crosslinkable group (such as a double bonding); or polymers having a structural feature in which a main chain bounds to side chains containing a crosslinkable function group capable of aligning liquid-crystalline molecules.

The polymers may be selected from polymers capable crosslinking themselves or polymers to be crosslinked by any crosslinkable agent, and such polymers may be used in any combination.

Examples of the polymer used for preparing an alignment layer include methacrylate copolymers described in the column [0022] in JPA No. Heisei 8-338913, styrene copolymers, polyolefins, polyvinyl alcohols, modified polyvinyl alcohols, poly(N- methylol acrylamide), polyesters, polyimides, vinyl acetate copolymers, carboxymethylcelluloses and polycarbonates. Silane coupling agents are also used as a polymer. Water-solbule polymers such as poly(N-methylol acrylamide), carboxymethylcelluloses, gelatins, polyvinyl alcohols or modified polyvinyl alcohols are preferred; gelatins, polyvinyl alcohols and modified polyvinyl alcohols are more preferred; and polyvinyl alcohols and modified polyvinyl alcohols are much more preferred. Using plural polyvinyl alcohols or modified polyvinyl alcohols, they have a different polymerization degree each other, is especially preferred.

The saponification degree of the polyvinyl alcohol is desirably from 70 to 100%, and more desirably from 80 to 100%. The polymerization degree of the polyvinyl alcohol is desirably from 100 to 5000.

In usual, the side chain having a function capable of aligning liquid-crystalline molecules may have a hydrophobic group as a function group. The types of the function group may be decided depending on various factors such as types of the liquid-crystalline compounds or desired alignment state. For example, the modified group can be introduced into the polyvinyl alcohol by copolymerization modification, chain-transfer modification or block-polymerization modification. Examples of the modified group include hydrophilic groups such as a carboxylic acid group, a sulfonic acid group, a phosphoric acid group, an amino group, an ammonium group, an amide group or a thiol group; C₁₀₋₁₀₀ hydrocarbon groups; hydrocarbon groups substituted with fluorine atoms; thioether groups, polymerizable groups such as an unsaturated polymerizable group, an epoxy group or an aziridile group; and alkoxysilyl groups such as tri-, di- or mono-alkoxysilyl group. Specific examples of such modified polyvinyl alcohols include those described in the columns [0022] to [0145] in JPA No. 2000-155216 and those described in the columns [0018] to [0022] in JPA No. 2002-62426.

It is possible to copolymerize a polymer in an alignment layer and a multi-functional monomer in an optically anisotropic layer, when the polymer in the alignment layer has a main chain bonding to side chains containing a crosslinkable functional group, or the polymer in the alignment layer has side chain being capable of aligning liquid-crystalline molecules and containing a crosslinkable functional group. In such case, not only between the multi-functional monomers but also between the polymers in the alignment layer and the multi-functional monomers and the polymers in the alignment layer, the covalent bondings are formed and the bonding strengths are improved. Thus, in such case, the strength of the optical compensatory sheet can be remarkably improved.

The polymer in the alignment layer desirably has crosslinkable functional group containing a polymerizable group. Specific examples include those described in the columns of [0080] to [0100] in JPA No. 2000-155216.

The polymer in the alignment layer may be crosslinked by using a crosslinkable agent.

Examples of the crosslinkable agent include aldehydes, N-methylol compounds, dioxane derivatives, compounds to act when being activated their carboxyl groups, active vinyl compounds, active halogen compounds, isoxazoles and dialdehyde starches. Single or plural type of crosslinkable agents may be used. Specific examples of the crosslinkable agent include the compounds described in the columns [0023] to [0024] in JPA No. 2002-62426. Aldehydes having a high reaction-activity are preferred, and glutaraldehydes are more preferred.

The amount of the crosslinkable agent is desirable from 0.1 to 20% by mass, and more desirably 0.5 to 15% by mass, with respect to the weight of the polymer. The residual amount of the unreacted crosslinkable-agent in the alignment layer is desirably not greater than 1.0% by mass, and more desirably not greater than 0.5% by mass. When the residual amount falls with in the range, the alignment layer has a sufficient durability, and even if the alignment layer is used in a liquid-crystal display for a long time, or is left under a high temperature and humidity atmosphere for a long time, no reticulation is appeared in the alignment layer.

The alignment layer may be prepared by applying a coating fluid, containing the above polymer, and, if necessary, the corsslinkable agent, to a surface of a support, drying under heating (crosslinking), and performing a rubbing treatment. The crosslinking reaction may be carried out any time after applying the coating fluid to a surface. When a hydrophilic polymer such as polyvinyl alcohol is used for preparation of an alignment layer, the coating fluid is desirably prepared using a mixed solvent of an organic solvent such as methanol, exhibiting a deforming function, and water. The weight ratio of water to methanol is desirably from 0/100 to 99/1, and more desirably from 0/100 to 91/9. Using such a mixed solvent can prevent bubbles from generating, and can remarkably reduce defects in the surface of the alignment layer and the optically anisotropic layer.

The coating fluid may be applied to a surface according to any known method such as a spin-coating method, a dip coating method, a curtain coating method, extrusion coating method, rod coating method, or roll coating method. The rod coating method is especially preferred. The thickness of the alignment layer after being dried is desirably from 0.1 to 10 micrometers. Drying may be carried out at 20 to 110° C. In order to form sufficient crosslinking, drying is desirably carried out at 60 to 100° C., and more desirably at 80 to 100° C. The drying may be continued for 1 minute to 36 hours, and desirably for 1 minute to 30 minutes. The pH is desirably set in a proper range for a crosslinkable agent to be used, and when glutaraldehyde is used, the pH is desirably set in a range from 4.5 to 5.5, and more desirably from 4.8 to 5.2.

The alignment layer may be formed on a surface of a support such as a polymer film or a surface of an under coating layer which is optionally formed on a support. The alignment layer can be obtained by applying a rubbing treatment to the surface of the polymer layer after crosslinking the polymer layer. Or the ability of controlling the alignment may be obtained by applying light-irradiation to the polymer layer.

The rubbing treatment may be carried out according to any known treatment used in a liquid-crystal alignment step of LCD. For example, the rubbing treatment may be carried out by rubbing the surface of a polymer layer with a paper, a gauze, a felt, a rubber, a nylon fiber, polyester fiber or the like in a direction. Usually, the rubbing treatment may be carried out by rubbing a polymer layer with a fabric in which fibers having a uniform length and line thickness are implanted averagely at several times.

The alignment treatment employing light-irradiation is carried out by irradiating a film formed on a substrate with light showing some kind of anisotropy, which obtains the ability of aligning liquid crystal composition. And the treatment may be carried out by using materials, described in “Liquid Crystal (Ekisyou)”, vol. 11, issue 1, p. 16 (2007); “Liquid Crystal (Ekisyou)”, vol. 8, issue 4, p. 216 (2004); “New Developments of Photo-functional Polymer Materials”, published by CMC in 1996, p. 167; and JPA No. 2007-156439, according to the methods described in these publications.

The alignment layer may be necessary for aligning the liquid crystal composition but, after fixing the liquid crystal composition in the alignment state, is not necessary and can be removed.

### [Support]

The optically anisotropic film of the invention may be formed on the support. The support is preferably transparent, and, in particular, preferably has a light transmission of not less than 80%. Examples of the support include polymer films. Examples of the polymer film which can be used as a support include cellulose ester films, polycarbonate films, polysulfone films, polyethersulfone films, polyacrylate films and polymethacrylate films. Cellulose ester films are preferable; acetyl cellulose films are more preferable; and triacetyl cellulose films are much more preferable. Polymer films produced according to a solvent casting method are preferable. The thickness of the support is preferably from 20 to 500 micro meters, and more preferably from 40 to 200 micro meters.

For providing the slip ability in the feeding step to the support, especially the long support, or for preventing any adhesion between the surface and the rear face in the rolled-up support, a polymer layer, containing inorganic particles having a mean particle size of 10 to 100 nm in a solid content concentration of 5 to 40%, may be formed on the one surface of the support according to a coating method or a multi-casting method.

The optically anisotropic film of the invention may have a sufficient adhesion to a glass substrate, a color filter formed thereon, alignment layer, OC layer, a reflection plate or the like. The adhesion strength may sometimes be necessary to be controlled, and in such a case, the adhesion strength can be controlled according to the method such as surface activation, surface modification and a method of using a coating fluid containing an agent capable of promoting the adhesion strength for preparing the optically anisotropic film of the invention.

In surface activation, the surface of the support or the like may be subjected to a chemical treatment employing acid or alkali, mechanical treatment, corona discharge treatment, flame treatment, UV treatment, high-frequency treatment, glow discharge treatment, active plasma treatment, ozone oxidation treatment, or the like. In the specification, the term “glow discharge treatment” is used for any treatments employing low-temperature plasma which is generated under a low-pressure gas of 10⁻³ to 20 torr and for any plasma treatments at an atmospheric pressure. The term “plasma excitation gas” means a gas which can be plasma-excited under the above mentioned condition; and examples of such a gas include argon gas, helium gas, neon gas, krypton gas, xenon gas, nitrogen gas, carbon dioxide gas, freon gas such as tetrafluoromethane and mixed gas of thereof. These matters are described in detail in Journal of Technical Disclosure No. 2001-1745, p. 30 to 32, issued on Mar. 15, 2001 by the Japan Institute of Invention and Innovation, and are preferably applicable to the present invention. For example, recently, a plasma treatment under an atmospheric pressure is receiving particular attention, and it may be carried out with an irradiation energy of 20 to 500 Kgy under 10 to 1000 Kev, and preferably with an irradiation energy of 20 to 300 Kgy under 30 to 500 Kev.

In surface modification, the surface of the support may be subjected to a forming treatment of an undercoating layer, silane-coupling treatment employing a silane-coupling agent, described hereinafter, forming treatment of an anchor-coating layer or the like. The undercoating layer is described in detail in Journal of Technical Disclosure No. 2001-1745, p. 32, issued on Mar. 15, 2001 by the Japan Institute of Invention and Innovation.

In a method of using a coating fluid containing an agent capable of promoting the adhesion strength for preparing the optically anisotropic film of the invention, an agent capable of promoting the adhesion strength is used, and examples of such an agent include those described in JPA Nos. Heisei 5-11439, Heisei 5-341532 and Heisei 6-43638. Specific examples of the agent include benzimidazoles, benzoxazoles, benzthiazoles, 2-mercapto benzimidazoles, 2-mercapto benzoxazoles, 2-mercapto benzthiazoles, 3-morpholinomethyl-1-phenyltriazole-2-thione, 3-molpholinomethyl-5-phenyl-oxadiazole-2-thione, 5-amino-3-molpholinomethyl-thiaziazole-2-thione, 2-mercapto-5-methylthio-thiadiazoles, triazoles, tetrazoles, benzotriazoles, carboxybenzotriazoles, amino-group-containing benzotriazoles, polymerizable organic metal compounds having a hydrolysable group or a radical-polymerizable group, α,β-ethylene-base unsaturated carboxylates, silane-coupling agents and titanium coupling agents. Examples of the silane-coupling agent include N-β-(aminoethyl)-γ-aminopropyl-trimethoxysilane, γ-methacryloxy-propyl-trimethoxysilane, vinyl trichlorosilane, vinyltris(β-methoxyethoxy)silane, vinyl triethoxysilane, vinyl trimethoxysilane, γ-aminopropyl triethoxysilane, γ-aminopropyl trimethoxysilane, γ-aminopropyl tripropoxysilane, γ-aminopropyl tributoxysilane, γ-aminoethyl triethoxysilane, γ-aminoethyl trimethoxysilane, γ-aminoethyl tripropoxysilane, γ-aminoethyl tributoxysilane, γ-aminobutyl triethoxysilane, γ-aminobutyl trimethoxysilane, γ-aminobutyl tripropoxysilane, and γ-aminobutyl tributoxysilane. Two or more species may be used in combination.

The amount of the agent capable of promoting adhesion is preferably from 0.001 to 20% by mass, more preferably from 0.01 to 10% by mass and even more preferably from 0.1 to 5% by mass with respect to the total amount of the ingredients (solid ingredients) used for preparing the optically anisotropic film.

A polymerization initiator may contribute to controlling the adhesion property. Examples of the polymerization initiator include halogenated hydrogen carbon derivatives such as the compounds having a triazine moiety or oxadiazole moiety; hexaarylbiimidazoles; oxime derivatives; organic peroxides; thio-compounds; ketones; acylphosphine oxides; aromatic onium salts; and methallocenes. Among these compounds, in terms of sensitivity, storage stability or adhesion strength, halogenated hydrocarbons having a triazine moiety, oxime derivatives, ketons and hexaarylbiimidazoles are preferable.

The optically anisotropic film of the invention is prepared by using a compound of the invention, and its retardation is expected to exhibit reversed wavelength dispersion characteristic at a visible-light wavelength region. Especially, retardation of the optically anisotropic film of the invention may exhibit reversed wavelength dispersion characteristics even at the longer wavelength region (for example, approximately 650 nm), although, previously, it is difficult to prepare a film having such characteristics. And so, the optically anisotropic film is useful as a wide λ/4 plate.

The novel compound of the invention and the film formed of a composition containing the compound is useful for preparing retardation plates and color filters exhibiting retardation.

The compound of the invention can be used as a dichromatic dye, and may also be used for preparing liquid crystal cells employing a guest-host mode and polarizing elements.

### EXAMPLES

Examples of the present invention will be explained below, without limiting the present invention.

### Example 1

### Exemplary Synthesis of Exemplary Compound (I-2)

Exemplary compound (I-2) was synthesized according to a route shown below.

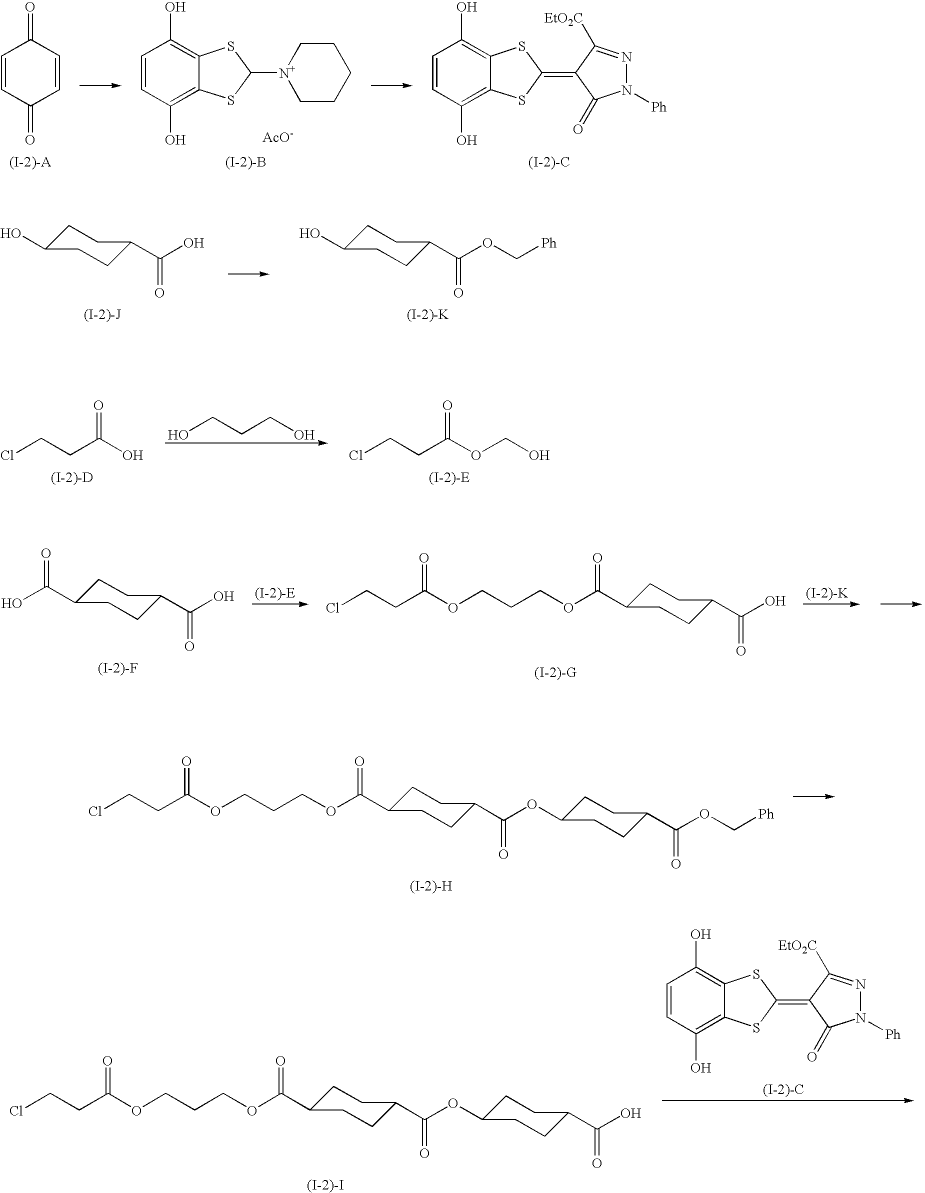

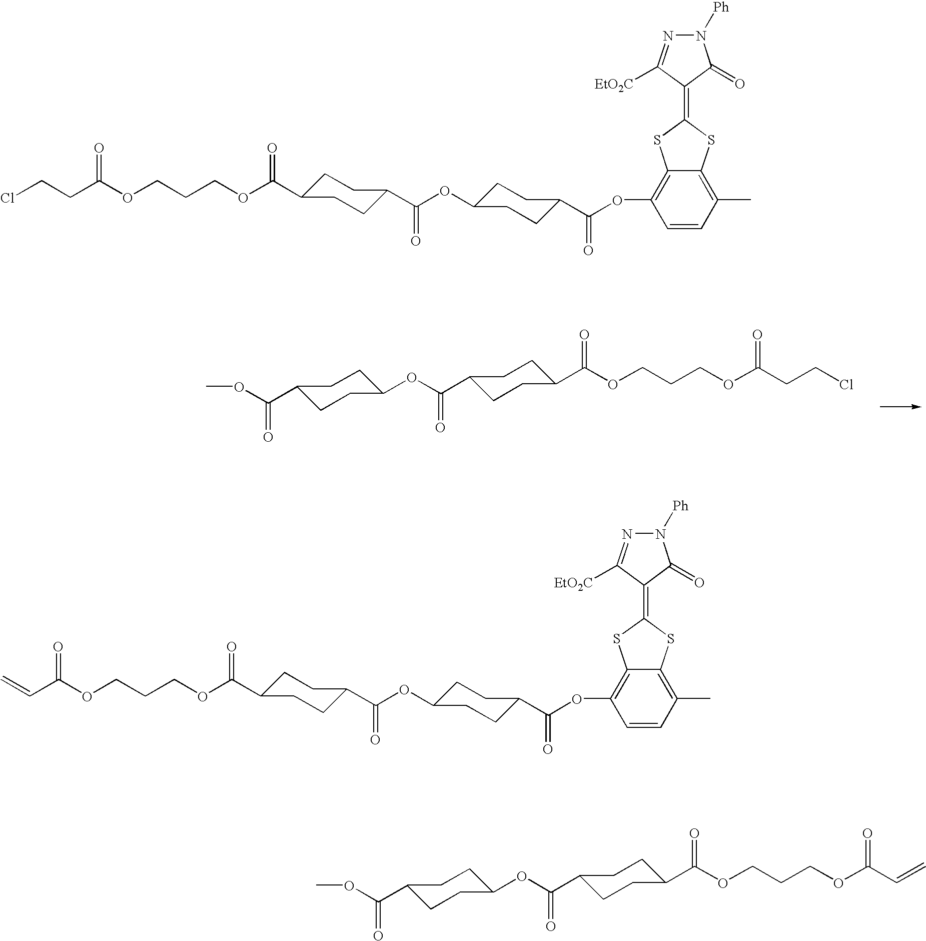

### Synthesis of Compound (I-2)-B:

Using benzoquinone (compound (I-2)-A) as a starting material, compound (I-2)-B was obtained according to the method described in J. Org. Chem., 69, p. 2164-2177 (2004).

### Synthesis of Compound (I-2)-C:

Added were 4.2 g (12.8 mmol) of compound (I-2)-B and 2.9 g (12.5 mmol) of ethyl 1-phenyl-2-pyrazoline-5-on-3-carboxylate to 50 mL of N-methyl-pyrrolidinone, and the mixture was stirred at 95° C. for 2 hours. The mixture was cooled to room temperature, added with 100 mL of ethyl acetate and 100 mL of water for extraction, the organic layer was washed with 100 mL of dilute hydrochloric acid, then washed with 100 mL of water, and dried over sodium sulfate. The obtained organic layer was condensed, and purified by silica gel column chromatography (ethyl acetate was used as an eluent), to obtain 3.2 g of the target compound (I-2)-C. The product was identified as the target substance by ¹H-NMR.

### Synthesis of Compound (I-2)-K:

Added were 7.2 g (50 mmol) of trans-4-hydroxy-cyclohexane carboxylic acid, and 10.2 g (60 mmol) of benzyl bromide to 90 mL of N-methyl-pyrrolidinone, the mixture was stirred, added with 8.2 g (100 mmol) of sodium hydrogen carbonate, and stirred at 90° C. for 3 hours. The mixture was cooled to room temperature, and added with 400 mL of ethyl acetate and 300 mL of water for extraction. The organic layer was washed with dilute hydrochloric acid and water, dried over sodium sulfate, and condensed. The condensate was purified by silica gel column chromatography, to obtain 9.2 g of the target compound (I-2)-K. The product was identified as the target substance by ¹H-NMR.

### Synthesis of Compound (I-2)-E:

Fifty-four grams (0.5 mol) of compound (I-2)-D was added to 500 mL of toluene 500 mL, the mixture was stirred, added with 38 g (0.5 mol) of 1,3-propanediol and 0.5 mL of sulfuric acid at room temperature, and refluxed under heating for 2 hours, while removing azeotropic water. The mixture was cooled to room temperature, added with 500 mL of water and 500 mL of ethyl acetate for extraction, the organic layer was dried over sodium sulfate, and then condensed. The condensate was purified by silica gel column chromatography, to obtain 41 g of compound (I-2)-E. The product was identified as the target substance by ¹H-NMR.

### Synthesis of Compound (I-2)-G

Thirty grams (0.17 mol) of trans-1,4-cyclohexane dicarboxylic acid (compound (I-2)-F) was added to 100 mL of toluene, the mixture was added with 0.5 mL of N,N-dimethylformamide, and 30 mL thionyl chloride at room temperature, and stirred under heating on an oil bath adjusted to 70° C. for 3 hours. The solvent was distilled off, the residue was added with 200 mL of tetrahydrofuran, added with 28.3 g (0.17 mol) of compound (I-2)-E at room temperature, the mixture was added dropwise with 14 mL (0.175 mol) of pyridine under cooling at 0° C. or below. The mixture was stirred at room temperature for 2 hours, added with 10 mL of pyridine, 400 mL of water, and 400 mL of ethyl acetate for extraction, the organic layer was dried over sodium sulfate, and condensed. The condensate was purified by silica gel column chromatography, to obtain 20 g (0.062 mol) of compound (I-2)-G. The product was identified as the target substance by ¹H-NMR.

### Synthesis of Compound (I-2)-H:

Twenty grams (62 mmol) of compound (I-2)-G was dissolved into 60 mL of toluene, the solution was added with 0.2 mL of N,N-dimethylformamide and 7.1 mL of thionyl chloride, and then stirred at 70° C. for 3 hours. The solvent was distilled off under reduced pressure, the residue was added with 100 mL of tetrahydrofuran, the solution was stirred, further added with 14.0 g (60 mol) of compound (I-2)-K, and the mixture was cooled to 5° C. or below. The mixture was sequentially added with 5 mL of pyridine under cooling, then added with 100 mg of 4-N,N-dimethylaminopyridine, the mixture was stirred at 5° C. or below for 30 minutes, and further stirred at room temperature for 2 hours. The reaction liquid was added with 200 mL of ethyl acetate and 200 mL of dilute hydrochloric acid for extraction, the organic layer was washed with water, dried over the sodium sulfate, and then condensed. The condensate was purified by silica gel column chromatography, to obtain 27.3 g (51 mmol) of the target compound (I-2)-H. The product was identified as the target substance by ¹H-NMR.

### Synthesis of Compound (I-2)-I:

Dissolved was 22.7 g (42 mmol) of compound (I-2)-H into 60 mL of tetrahydrofuran and 60 mL of ethanol, the solution was added with 2 g of 10% Pd—C, and then stirred under a hydrogen atmosphere (5 Mpa) at room temperature for 3 hours. The catalyst was removed by filtration, and the solvent was distilled off to obtain 17.7 g (40 mmol) of the target compound (I-2)-I. The product was identified as the target substance by ¹H-NMR.

### Synthesis of Compound (I-2)-J:

Dissolved was 5.2 g (12 mmol) of compound (I-2)-I into 20 mL of toluene, the solution was added with 0.1 mL of N,N-dimethylformamide and 1.3 mL of thionyl chloride, and then stirred at 70° C. for 3 hours. After the solvent was distilled off under reduced pressure, the residue was added with 20 mL of N-methyl-pyrrolidinone, stirred, further added with 2 g (4.8 mol) of compound (I-2)-C, and then cooled to 5° C. or below. The solution was sequentially added with 1 mL of pyridine and 100 mg of 4-N,N-dimethylaminopyridine under cooling, stirred at 5° C. or below for 30 minutes, and further stirred at room temperature for 2 hours. The reaction liquid was added with 200 mL of ethyl acetate and 200 mL of dilute hydrochloric acid for extraction, the organic layer was washed with water, dried over magnesium sulfate, and concentrated. The concentrate was purified by silica gel column chromatography, to obtain 2.8 g (2.2 mmol) of the target compound (I-2)-J. The product was identified as the target substance by ¹H-NMR.

Thus-synthesized compound (I-2)-J showed phase transition temperatures below:

Crystal 140° C.→Nematic phase 178° C.→Isotropic phase

### Synthesis of Compound (I-2):

Dissolved was 2.54 g (2 mmol) of compound (I-2)-J into 20 mL of acetonitrile, the solution was added with 0.7 mL of triethylamine, and then stirred at 80° C. for 3 hours. The solvent was distilled off under reduced pressure, and the concentrate was purified by silica gel column chromatography, to obtain 1.9 g (1.6 mmol) of the target compound (I-2). The product was identified as the target substance by ¹H-NMR.

NMR data obtained from the measurement are shown below.

¹H-NMR (300 MHz, CDCl₃): 8.01 ppm (2H,d), 7.45 ppm (2H,t), 7.28 ppm (1H,d), 7.26 ppm (2H,d), 6.43 ppm (2H,d), 6.14 ppm (2H,dd), 5.85 ppm (2H,dd), 4.80 ppm (2H,m), 4.51 ppm (2H,q), 4.20 ppm (4H,t), 4.15 ppm (4H,t), 2.70 ppm (2H,m), 1.4-2.4 ppm (39H,m).

Thus-synthesized compound (I-2) showed phase transition temperatures below:

Crystal 145° C.→Nematic phase 185° C.→Isotropic phase

### Example 2

Compounds (I-1), (I-3) to (I-5), (I-11), (I-13), and (I-16) were respectively synthesized by replacing, (I-2)-I in the synthetic route in Example 1, with any of correspondent I-Substance, and replacing (I-2)-C with any of correspondent C-Substance listed below.

| I-Substance | C-Substance |
|---|---|
| 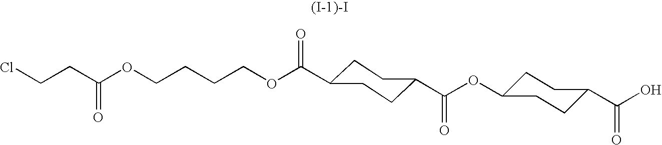 | 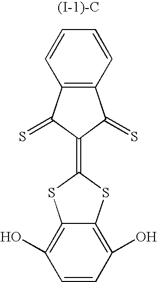 |
| 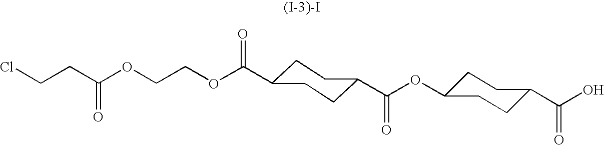 | 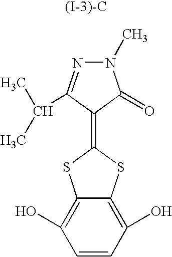 |
| 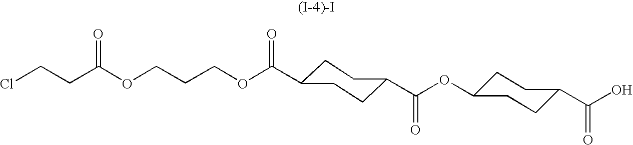 | 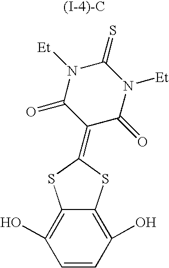 |
| 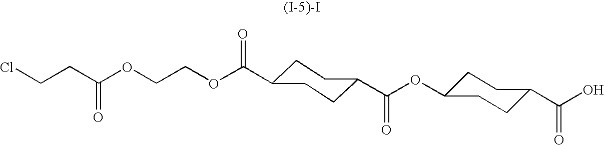 | 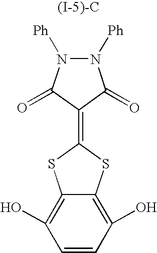 |
| 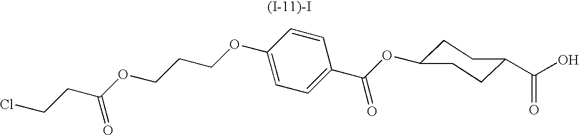 | 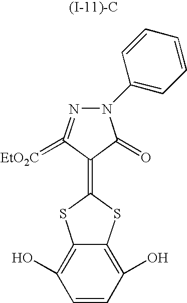 |
| 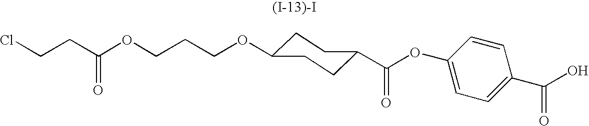 | 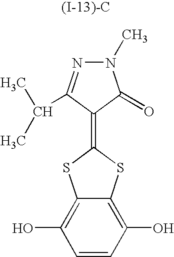 |
| 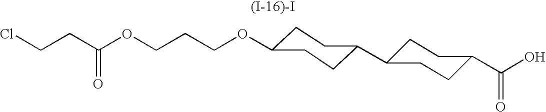 | 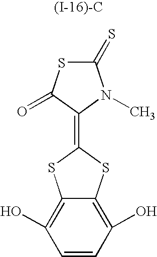 |

### Example 3

### Measurement of Absorbance of Solution

Five milligrams of compound (I-2) was dissolved into 250 mL of chloroform, and absorption spectrum over the UV to visible regions was measured. λmax was observed at 427 nm, with a molar absorption coefficient of 2×10⁴.

### Example 4

### Monoaxial Alignment

Twenty milligrams of compound (I-2) was dissolved in 40 mL of chloroform, and spin-coated on a glass substrate preliminarily rubbed. The substrate was dried at 170° C. for one minute, and observed under a polarizing microscope. Molecules of compound (I-2) were found to uniformly aligned. The slow axis was found to coincide with the direction of rubbing, and the field of view alternately turned bright and dark every 45-degree rotation of the stage.

### Example 5

### Wavelength Dispersion Characteristics of Birefringence

Compound (I-2) was introduced into a wedge-form cell, and birefringence (Δn) was observed under heating. Results are shown in Table 1. For reference, also a referential compound-1 having a structure below was similarly observed, and results were shown.

| | Birefringence (Δn) at Measurement Wavelengths | | | | |
|---|---|---|---|---|---|
| | 450 nm | 500 nm | 550 nm | 600 nm | 650 nm |
| Compound | | 0.025129 | 0.031024 | 0.033649 | 0.034815 |
| (I-2)-J | | | | | |
| Referential | 0.035192 | 0.039614 | 0.041104 | 0.042235 | 0.042533 |
| Compound 1 | | | | | |

Characteristics of birefringence was determined from values of birefringence at the individual wavelengths obtained by the measurement, and listed in the Table 2 below. Table 2 shows, for reference, also values of ideal wavelength dispersion characteristics showing wide-band performance. These values are determined based on ratio of values of birefringence at different measurement wavelengths. For example, as for Δn(450 nm)/Δn(550 nm), an ideal value was determined by dividing one measurement wavelength 450 nm with the other measurement wavelength 550 nm (450/550=0.82)

| | Wavelength Dispersion Characteristics | | |
|---|---|---|---|
| | of Birefringence (Δn) | | |
| | Δn(450 nm)/ | Δn(500 nm)/ | Δn(550 nm)/ |
| | Δn(550 nm) | Δn(600 nm) | Δn(650 nm) |
| Compound | — | 0.747 | 0.891 |
| (I-2)-J | | | |
| Referential | 0.856 | 0.938 | 0.966 |
| Compound 1 | | | |
| Ideal value*1 | 0.82 | 0.83 | 0.85 |

*1Ideal wavelength dispersion characteristics showing wide-band performance

From the results shown in Table 2, it is understood that Compound (I-2)-J, the compound of the present invention, shows values closer to the ideal wavelength dispersion characteristics of retardation in the longer wavelength regions, as compared with Referential compound 1.
Referential Compound 1 (a compound described in Japanese Unexamined Patent Publication No. 2007-318886)

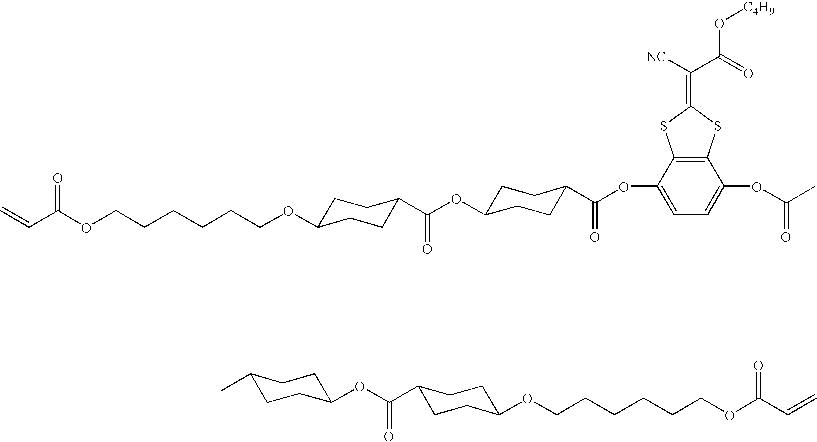

What is claimed is:

1. A compound represented by formula (I) below: 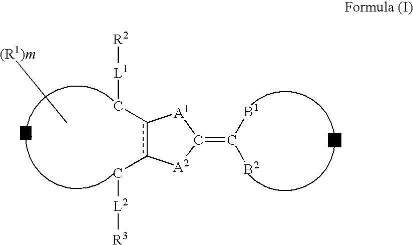
where, each of A¹ and A² independently represents a group selected from the group consisting of —O—, —NR— (R represents a hydrogen atom or substituent), —S— and —CO—; Z represents one or two atoms selected from the group consisting of carbon atom and the Group XIV to Group XVI non-metallic atoms, and forms a five- or six-membered ring together with C—C═C—C or C═C—C═C illustrated in the formula; each of R¹, R² and R³ independently represents a substituent; m represents an integer from 0 to 4; each of L¹ and L² independently represents a single bond or divalent linking group; each of B¹ and B² independently represents a group selected from the group consisting of —O—, —S—, —SO—, —SO₂—, —NR— (R represents a hydrogen atom or substituent), ═N—, ═N⁽⁺⁾R— (R represents a hydrogen atom or substituent), —CO—, —CS—, and ═CR— (R represents a hydrogen atom or substituent); Y represents two to four atoms selected from the group consisting of carbon atom and the Group XIV to Group XVI non-metallic atoms, and forms a five- to seven-membered ring together with B¹—C—B² illustrated in the formula, allowing thereon bonding of substituent R⁴, provided that those having the same substituent for A¹ and B¹ and for A² and B² and those having the same substituent for A¹ and B² and for A² and B¹ are excluded.

2. The compound of claim 1, represented by formula (I)′ below: 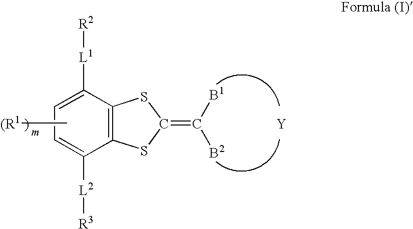
where, S represents a sulfur atom, and definitions of all other symbols are same as those defined in formula (I).

3. The compound of claim 1, wherein the ring Y is represented by any of formulae Y-1 to Y-24 below: 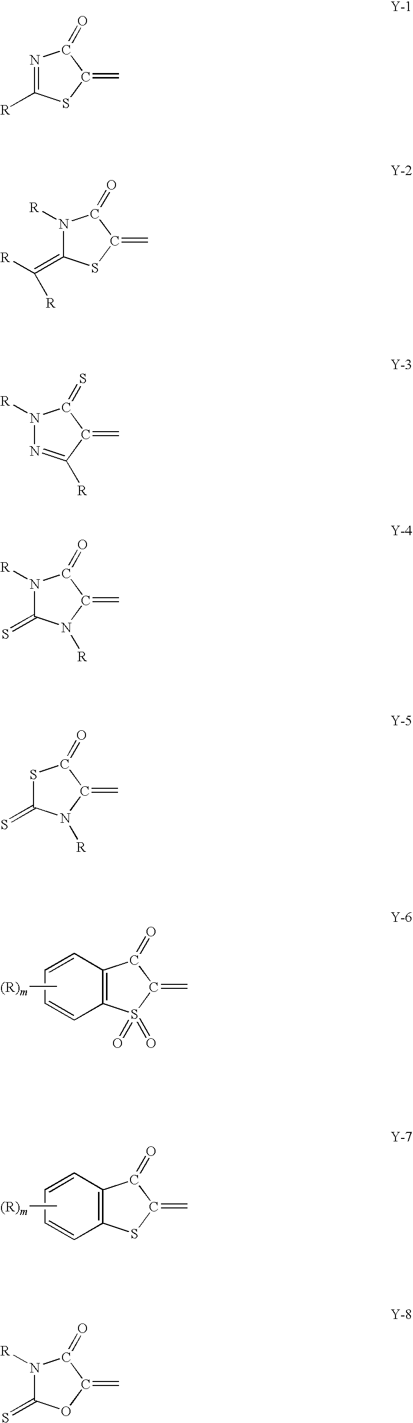 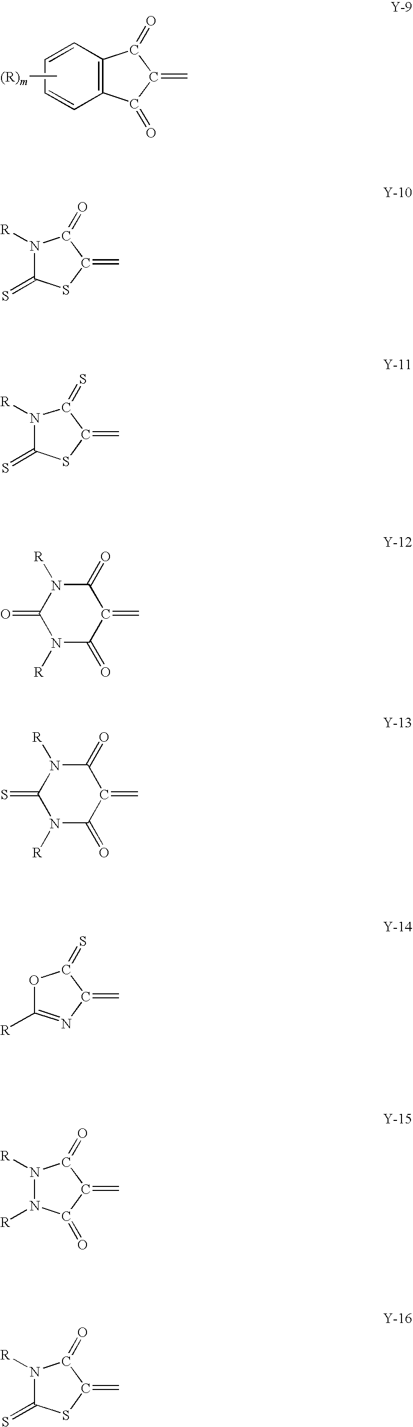 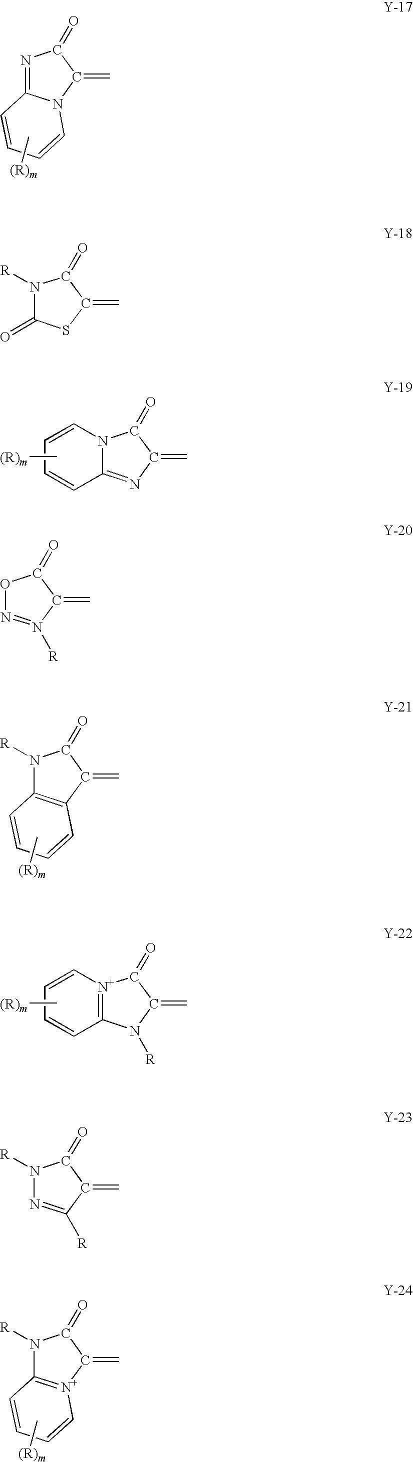
where, R represents a hydrogen atom or substituent; a plurality of (R)s, if coexist therein, may be same with, or different from each other; and m represents an integer from 0 to 4.

4. The compound of claim 1, represented by any one of the formulae (I-a) to (I-e) below: 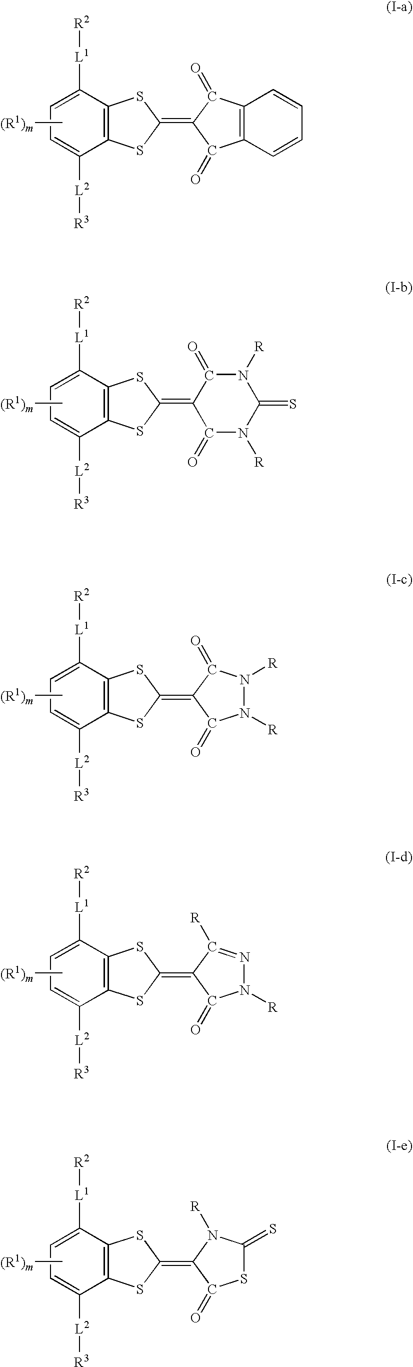
where, S represents a sulfur atom, definitions of all other symbols are same as those defined in the formula (I), R represents a hydrogen atom or substituent; and a plurality of (R)s, if coexist therein, may be same with, or different from each other.

5. The compound of claim 1, wherein at least either one of L¹ and L² in formula (I) represents —OCO—.

6. The compound of claim 1, wherein at least one of R, R¹, R², R³ and R⁴ in formula (I) represents a substituent containing polymerizable group(s).

7. The compound of claim 1, wherein each of R² and R³ is represented by any one of the formulae below: 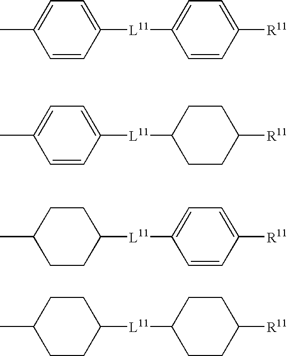
where, L¹¹ represents a single bond or linking group; and R¹¹ represents a substituent.

8. The compound of claim 1, wherein each of R² and R³ is represented by any one of the formulae below: 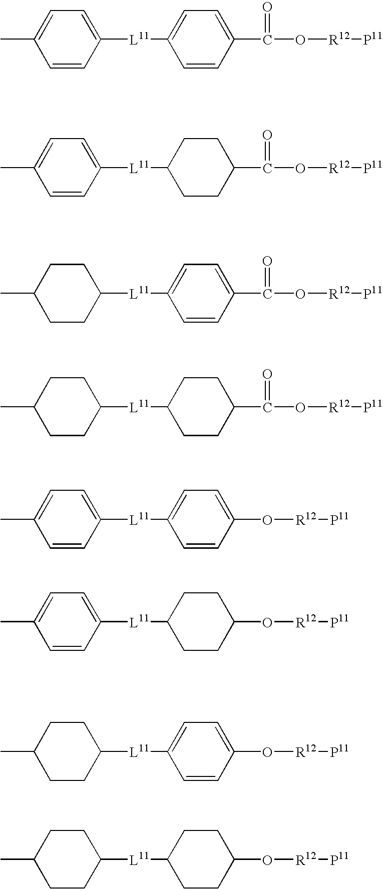
where, L¹¹ has a same definition defined as in the above; R¹² represents an alkyl group (where, either one of non-adjacent carbon atoms may be substituted by an oxygen atom or sulfur atom); and P¹¹ represents a polymerizable group.

9. A liquid crystal composition comprising at least one species of a compound as set forth in claim 1.

10. An optically anisotropic material formed of a composition as set forth in claim 9.
